(19) United States
(12) Patent Application Publication
(10) Pub. No.: US 2026/0041346 A1
(43) Pub. Date: Feb. 12, 2026

(54) AUDIO-BASED INTERACTIVE SIMULTANEOUS EXERCISE AND COGNITIVE CONTROL TRAINING FOR IMPROVING COGNITIVE NETWORKS SUBSERVING NEUROPSYCHIATRIC AND NEUROCOGNITIVE CONDITIONS

(71) Applicants: Sarah McEwen, San Diego, CA (US); Robert McEwen, San Diego, CA (US)

(72) Inventors: Sarah McEwen, San Diego, CA (US); Robert McEwen, San Diego, CA (US)

(21) Appl. No.: 19/362,386

(22) Filed: Oct. 18, 2025

Related U.S. Application Data
(63) Continuation of application No. 17/398,966, filed on Aug. 10, 2021.

(51) Int. Cl. A61B 5/16; A61B 5/00; G16H 20/70; G16H 50/70

(52) U.S. Cl. CPC A61B 5/168; A61B 5/0004; A61B 5/4088; A61B 5/6898; A61B 5/7405; A61B 5/746; A61B 5/7475; G16H 20/70; G16H 50/70

(57) ABSTRACT

A method and system utilize unexpected combinations of cognitive exercises to provide routines performed simultaneously with aerobic exercise to address cognitive impairments. Cognitive exercises comprise specific steps in order to interact with a specific cognitive domain. All stimuli may be aural. Specific combinations of cognitive exercises are created to engage selected brain circuits and are correlated to specific cognitive impairments. The method may remediate selected cognitive impairments and strengthen cognitive abilities. The user responds to instructions for each exercise and performs each exercise in a specific order for a specific duration of time and through a specific number of sets. A processor performs a program comprising the routines and evaluates performance of a user. A program may be customized for an individual user. An individual user's data is processed to evaluate progress. Large libraries of data for multiple users may be processed to provide statistical databases.

### FIELD

The present disclosure relates generally to a system and method simultaneously utilizing aerobic exercise and neuroscience-based cognitive training programs that may each be targeted to strengthen a cognitive ability through engaging underlying neural circuits.

### BACKGROUND

Age-related cognitive decline is a growing problem affecting the increasing numbers of older people in the population. Cognitive decline results in reduced mental clarity and results in decreased cognitive executive functions. It also impacts physical ability and overall health. Cognitive impairments that may not be age related, e.g., depression, can also impair day to day human functioning. This includes comprehending medication labels and responding to actionable information. Physical daily life tasks, such as driving, are also affected. A consequence of physical aging is that the human brain ages and cognitive abilities are affected. A common consequence of reduced mental functioning is dementia. Age-related memory decline results from exponential neuronal loss and impaired replacement of the lost cells. Cognitive impairment is a growing societal problem. For example, the World Health Organization reveals that 47.5 million people worldwide have dementia with an annual incidence of 7.7 million new cases every year. This number is projected to increase to 75.6 million people by 2030.

Memory decline occurs through various mechanisms. Cognitive decline is progressive, but generally does not proceed at a linear rate. Higher level functions are affected by age. Not all areas decline. Some cognitive functions are performed just as well as performed by younger people. The degree of decline and the areas in which the decline occurs varies among individuals. Variation is due to individual physical makeup, lifestyle, and environment. Decrease in executive function is a key contributor to age-related cognitive decline and is seen in performance in a range of cognitive tasks. Slowing of declines may be beneficial because deficits of early cognitive processing stages can affect cognitive functions in later processing stages.

Daily life is greatly affected by the ability to perform cognitive skills and physical skills simultaneously. Performance in dual tasks including walking and processing information or talking is necessary for successfully carrying out activities of daily living. Many approaches have been taken to improve cognitive abilities or to delay the onset of further cognitive decline. These methods have included mental exercises. Mental exercises have had limited success at best, due to the inability to transfer these trained skills to more general everyday functioning activities. More recently the prior art has begun to explore the use of both cognitive training and motor training.

The prior art has shown that one of the best ways to increase cognitive reserve and reduce effects of aging is through physical exercise-induced neuroplasticity. Combinations of aerobic exercise and memory training in the prior art have been formulated for the purpose of improving mental performance of adults. The objective is to delay subjective cognitive decline (SCD), which is memory loss or decline in cognitive performance. These factors are early signs of Alzheimer's disease. The prior art has been substantially limited to delaying the onset of Alzheimer's disease. Currently, Alzheimer's disease is not treatable. The prior art methods focus on the delaying or preventing of the onset of dementia. Dementia is a general term for a decline in mental ability severe enough to interfere with daily life. Alzheimer's is the most common cause of dementia. Alzheimer's is a specific disease. These efforts focused on preventing the onset of dementia have been generally directed to prevention of Alzheimer's disease. The literature is predominantly directed toward Alzheimer's disease rather than to other cognitive impairments. A large body of literature is primarily directed to dealing with SCD. In addition, patents and published patent applications address dealing with cognitive function. There are many conditions that affect cognitive decline in addition to Alzheimer's. These include PTSD, ADHD, brain fog, depression, autism, cognitive aging, and OCD.

Human and animal studies demonstrate that exercise affects biochemical, neurological, and behaviors in human and animal studies. Mechanisms of enrichment of the brain's neurochemical environment include: neurogenesis, i.e. production of new neurons; cell proliferation; angiogenesis, i.e. production of new blood vessels; synaptic protein expression, i.e. synapsis-resistance; cerebral blood flow; production of growth factors and nutrients including BDNF, VEGF, IGF-1; reduction of inflammation; and increased function of neurotransmitters.

A current apparatus used to provide simultaneous physical and cognitive exercise is the CyberCycle Bike. Current retail price of this apparatus is approximately $9000.00. This price is prohibitive for many clinical treatment locations, gyms, and individuals. This apparatus requires engaging in a virtual reality tour. Flexibility in applications is not provided.

The prior art discloses exercise programs that include both physical and cognitive exercises. In one program physical exercise is performed prior to cognitive exercises. Prior art has shown that separation of performance of physical and cognitive tasks is not optimal. Many simultaneous aerobic exercise and memory training programs have been provided in a number of prior studies. These programs utilized exercises which consisted of fixed steps in sequence. These sequences were used individually and were not tailored to specific patients or conditions.

U.S. patent application No. 20160293033 by Anderson-Hanley discloses a system and method for the purpose of improving cognitive function of an individual. The individual uses stationary exercise equipment, such as a stationary bicycle, and faces a display. The individual propels an avatar on the display through a representation of a pathway. The user is presented with a first set of stimuli and must respond. Successive presentations of the first set of stimuli are repeated until the cognitive task is successfully completed a predetermined number of times. Further sets of stimuli are provided. This method and system require a computer-based video program which provides an essentially fixed environment for the cognitive exercises. The user is confined to one physical space. The user's attention must be continuously focused on the display. This results in the user's being unable to focus on other visual stimuli. It may be difficult for many individuals with cognitive impairments to focus continually on a display and also to perform functions as a result of the stimuli. This method requires the use of apparatus which may be expensive. Also a technician or aide may be required for physical assistance.

U.S. patent application No. 20180261115 by Gazzaley discloses a method in which a user operates exercise apparatus and is given cognitive tasks to perform. As the tasks are performed, the user is presented with distractions such as interference or interruptions and must complete the tasks in the presence of the distractions. User reactions are measured to detect improvement in performance. This process measures an individual's ability to ignore the interruption and continue with the assigned task to be performed. An interrupter is to be responded to as a secondary task. This is specifically directed toward causing an individual to respond to multiple stimuli rather than to performing a single task. This method utilizes a distraction rather than the performance of physical exercise.

U.S. Pat. No. 10,593,221 to Johnson et al. discloses an audio-only interference training system. First and second audio sources provide a task signal and an interference signal. An individual must respond to a defined task based on the two audio signals. This is an interference method and does not utilize aerobic exercise. The benefits of performing at an increased heart rate cannot be obtained.

U.S. patent application No. 20190351289 discloses stimulation of neurogenesis by aerobic exercise with virtual reality enhanced brain function tasks. The user performs bodily movement on an exercise machine. The user is also presented with tasks to perform in a virtual reality environment. Recovery of damaged competence in targeted brain functions is achieved by use of physical equipment and making use of immersive virtual or augmented reality games. This system requires fixed equipment which may be expensive and is not easily deployed in more than one location.

### SUMMARY

Briefly stated, the present subject matter provides audio-based interactive simultaneous exercise and cognitive control training for improving cognitive networks subserving mental health and neurocognitive conditions. Exercises train cognitive abilities of a user that have been disrupted. These primary cognitive abilities include memory, attention, decision making, and processing of information.

The method utilizes “top-down” training. In other words, exercises invoke executive functioning in the cognitive training. A suite of training routines is provided. Each training routine comprises a selection of exercises ordered in a sequence. The cognitive exercises presented during the exercise sessions theorized to engage top-down executive functioning processes. Top-down cognitive control engages a range of executive function tasks, which are central to most neuropsychiatric disorders. Cognitive flexibility is the ability to change one's behavior according to contextual cues. Inhibitory control is not a unitary function and consists of motor (or behavioral) response inhibition and interference control (or cognitive inhibition). Behavioral inhibition involves the inhibition of pre-potent and automatic motor responses. Cognitive inhibition, on the other hand, refers to the cognitive control needed to prevent interference due to competition by irrelevant stimuli or irrelevant stimulus characteristics. Whereas the cognitive tasks in each exercise program are chosen to train the most significant functions for the selected clinical or cognitive condition being addressed.

Cognitive rehabilitation training using neuroscience-based cognitive tasks is performed while the user simultaneously performs physical exercise. New applications for the most effective type of brain-body training are provided. These applications include selected combinations of types of exercises and duration and sequence of exercises. Through brain performance self-assessment or via the users practitioner, programs are individualized and calibrated to promote optimal brain health and to help users achieve higher levels of cognitive performance.

The range of cognitive impairments that can be addressed is vastly expanded. Aerobic exercise, which elevates the heart rate to a moderate intensity level (optimal for neuroplasticity-based enhancements), is preferred. An interface is provided to couple the user to stimuli coming from a cognitive training exercise program. All stimuli or a predominant body of stimuli are aural. Auditory stimuli engage the neural networks associated with particular cognitive impairments. While a user performs physical exercise, the user also performs cognitive tasks in accordance with audible instructions from a processor.

Prior art methods and systems are limited in application to cognitive impairments. In accordance with the present subject matter, by establishing synergistic combinations of cognitive exercises the capabilities of cognitive rehabilitation are vastly expanded. The present subject targets neural networks in the brain. Specific exercises are utilized to target specific cognitive disabilities. This allows the exercises to impact and improve specific cognitive impairments, such as ADHD, PTSD, brain fog, depression, and Parkinson's Disease.

### BRIEF DESCRIPTION OF THE DRAWINGS

FIG. 1 is an illustration of a user employing the present matter;

FIG. 2 is an illustration of areas of the brain which operate to initiate functions such as neurogenesis and angiogenesis;

FIG. 3 is an illustration of various lobes in the brain and cognitive skills associated with the lobes;

FIG. 4 is a diagram of neural circuits underlying cognitive domains;

FIG. 5 is a block diagram of apparatus used in performance of the current method;

FIG. 6 is a timing chart showing the sequence of a nominal cognitive exercise in which the abscissa is time;

FIG. 7 illustrates content delivery to a user performing an exercise;

FIG. 8 is a graphical user interface through which the user cooperates with the program;

FIG. 9 is a flowchart illustrating performance of the program;

FIG. 10 is a chart of cognitive exercises and a description of parameters of the exercise and the portions of the brain engaged;

FIG. 11 illustrates a nominal program individualized to a user;

FIG. 12 illustrates a comprehensive neurocognitive testing assessment; and

FIG. 13 is a chart of clinical disorders and the neural circuits associated with each disorder.

### DETAILED DESCRIPTION

The present subject matter provides significant advances over prior art methods which combine physical exercise with neurocognitive training tasks. A method and system train cognitive abilities of a user that have been disrupted. Capabilities extend beyond the mere ability to attempt to delay neurocognitive aging. Memory, attention, reasoning, and information speed of processing are foundational cognitive domains trained in the current method and system. Normal aging causes reduction in neuroplasticity along with resulting decreases in cognitive ability. It has been found that neuroplasticity can be improved through a combination of physical exercise and cognitive tasks.

The present method and system provide cognitive training with the capability of addressing individual areas of cognitive ability, also referred to as cognitive functions. Cognitive functions for purposes of the present description include normal cognitive functions and impaired cognitive functions. This method activates specific neural circuits for engaging cognitive functions by instructing a user to perform a set of designated cognitive skill routines and ordering the designated cognitive skill exercises within a routine. Ordering the designated cognitive skill exercises comprises selecting a time period for performing exercises and providing one of a preselected set of routines and establishing a preselected time duration for performing each exercise. Each cognitive skill exercise is associated with a selected cognitive skill. Each cognitive skill exercise is defined by a sequence of steps and a number of repetitions of the sequence of steps. The method provides instructions to a user for each cognitive exercise in a routine. The method provides interactive stimuli, registers the user's response to stimuli, and provides outputs indicative of responses of the user. In one preferred form each sequence of steps provides aural stimuli. Comparing the outputs to preselected criteria provides a measure of the user's performance based on the comparing of outputs. The method provides for establishing a routine which comprises creating a set of cognitive exercises which when taken together address a respective cognitive impairment or address a set of skills for use by a preselected population.

The present method and system provide cognitive training with the capability of addressing individual areas of cognitive ability. This will provide the first mobile-based, simultaneous aerobic exercise and cognitive remediation program for mental health, neurocognitive and neurodegenerative conditions. This method and system are performed using an apparatus allowing interaction with a user. A cognitive training application is preferably housed in a program memory. The program memory may be located in a smartphone or in a remote server. The interface comprises transducers enabling communication between the user and the program. Transducers may include a headphone or speaker providing audio stimuli to the user and a keypad allowing the user to provide inputs to the cognitive training program. The transducers and program memory are preferably included in a smartphone.

This program provides mobile audio-based interactive simultaneous exercise and cognitive control training for improving cognitive networks subserving mental health and neurocognitive conditions. Exercises train cognitive abilities of a user that have been disrupted. These abilities include memory, decision making, and processing of information.

PTSD affects memory, attention, planning, and problem solving. ADHD causes weakness in executive functioning. Brain fog inhibits focusing on executive function and causes confusion and impairs working memory. Depression impairs attention and memory, as well as information processing and decision-making skills. It can also lower cognitive flexibility, the ability to adapt goals and strategies to changing situations, and executive functioning. Cognitive aging causes numerous symptoms including memory impairment, loss of sense of direction, confusion, and difficulty with personal care. Executive function relates to abilities to differentiate among conflicting choices, determine future consequences of current activities, working toward a defined goal, prediction of outcomes, and social cognition. The present method and system address these issues through simultaneous performance of physical exercise and cognitive exercises.

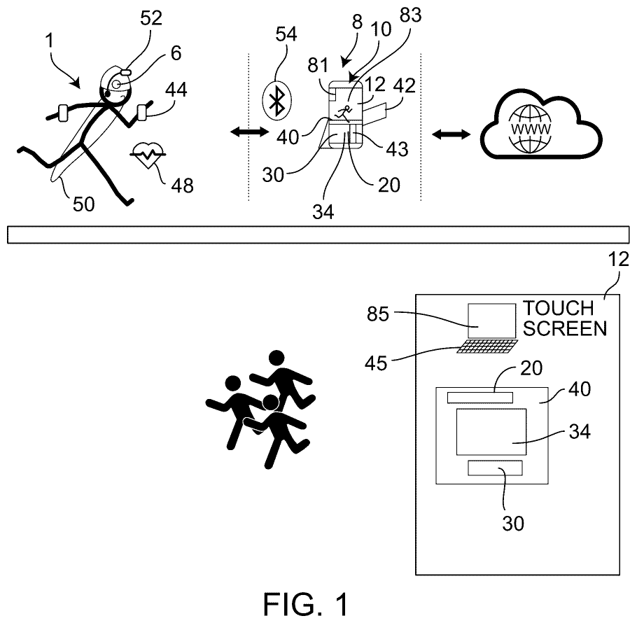

FIG. 1 is an illustration of a user 1 employing the present matter to perform physical exercise in combination with neurocognitive training tasks to improve cognitive functioning in a brain 6 of the user 1. The method addresses cognitive skills performed utilizing cognitive skill development simultaneously with physical exercise. The method of the present disclosure embodies interaction of the user 1 with a digital interactive device 10 which may be portable. In one preferred form the digital device 10 is a portable interactive device comprising a smartphone 12. The system of the present disclosure comprises the physical apparatus which may be used to practice the method. The method comprises providing programs including routines, the routines including exercises. The routines are each associated with a cognitive skill, providing instructions to a user to perform certain cognitive tasks, registering the user's response, processing data including the user's response, and generating process data indicative of parameters relating to cognitive skills. The method may further include generating a database comprising a plurality of results for one user and data for populations of users. Neurocognitive training tasks are provided for improving processing speed, executive functioning, memory, and attention. A cognitive training program 20 is executed by a cognitive training program app 30. The app 30 is accessed from a program memory 34 in a processor 40 included in the smartphone 12. The smartphone 12 provides an interface between the user 1 and the training program 20.

In the illustration of FIG. 1, the user 1 is performing physical exercise by running. The user 1 receives audio stimuli from the smartphone 12. Because the stimuli are aural, the user 1 can pay full attention to physical surroundings and may provide responses to stimuli comprising touch on a data input 42 or motion sensor 44, which may include a touch input. The data input 42 may comprise a touch screen 83 or a keyboard 43. This system can be manufactured at a low cost compared to other cognitive training systems, such as the CyberCycle Bike which runs approximately $9000.00. Optional components may be employed in addition to the smartphone 12. The motion sensor 40 is a 3D motion tracking sensor. An augmented reality (A/R) headset 52 may be used to provide further context to the user's unaided view. The heart rate monitor 48 may comprise a Bluetooth-coupled heart rate sensor.

The intensity of physical exercise is monitored by a heart monitor 48. The user 1 may employ headphones 50 coupled by a Bluetooth circuit 54 to the smartphone 12. Alternatively, audio may be provided by a speaker 81 in the smartphone 12 The neurocognitive tasks promote improvements in processing speed, executive functioning, memory, and attention.

The benefits of physical exercise in conjunction with cognitive training have been demonstrated and described in many different journal articles. One significant article describing this phenomenon is McEwen, et al., Simultaneous Aerobic Exercise and Memory Training Program in Older Adults with Subjective Memory Impairments, Journal of Alzheimer's Disease 62 (2018) 795-806. As of the time of this study, there were no currently effective treatments for dementia. Therefore, preventive strategies to delay or prevent the onset of dementia were of critical importance. This study had the objective of determining the relative effectiveness of simultaneous performance of memory training and aerobic exercise to a sequential performance intervention on memory functioning in older adults. The subjects included older adults aged 60-75 with subjective memory impairments. They had supervised strategy-based memory training done simultaneously while stationary cycling or sequentially after stationary cycling. The study found that a four-week simultaneous memory training and aerobic exercise program was sufficient to improve memory, attention, and reasoning abilities in older adults.

Simultaneous cognitive-motor training is defined as training where both motor training and cognitive training are performed at the same time. Much of everyday activities such as the dual tasks of walking while engaged in discussion depend on the ability to attend to competing actions and then balance system demands by switching to the most task-relevant information as it becomes available. In a successful combination of cognitive-motor training, the cognitive task is incorporated into the physical motor task. This type of cognitive-motor training is often referred to as moving while thinking. Aerobic or cardiovascular-based exercise: is a system of conditioning aimed at enhancing circulatory and respiratory efficiency that improves the body's use of oxygen through sustained exercise.

The method of this system utilizes “top-down” cognitive control through simultaneous exercises and cognitive stimulation wherein the cognitive exercises are selected and performed during exercise with a response decision required to engage top-down executive cognitive control skills. The top-down concept is important. This addresses executive functioning. The overarching concept is cognitive control training. Top-down training is new in the present context. Executive functioning, attention, explicit memory, and coordination with physical movement are rely on top-down cognitive abilities. Top-down cognitive control engages a range of executive function tasks, which are central to most neuropsychiatric disorders and provide a generalized method to improve cognitive functioning in one's daily life.

The usage of physical exercise during cognitive training invokes further engagement of executive functioning abilities during the cognitive training sessions. A suite of training routines is provided. Each training routine comprises a selection of exercises ordered in a sequence and each exercise in the program is specifically chosen to complementarily strengthen cognitive networks. New applications for the most effective type of brain-body training are provided. These applications include selected combinations of types of exercises and duration and sequence of exercises. Through brain performance assessment, programs are individualized and calibrated to promote optimal brain health and to help users achieve higher levels of cognitive performance. Different physiological and biological effects have been found to be specifically associated with skill-based, as opposed to more automatic and non-cognitively demanding, physical exercises.

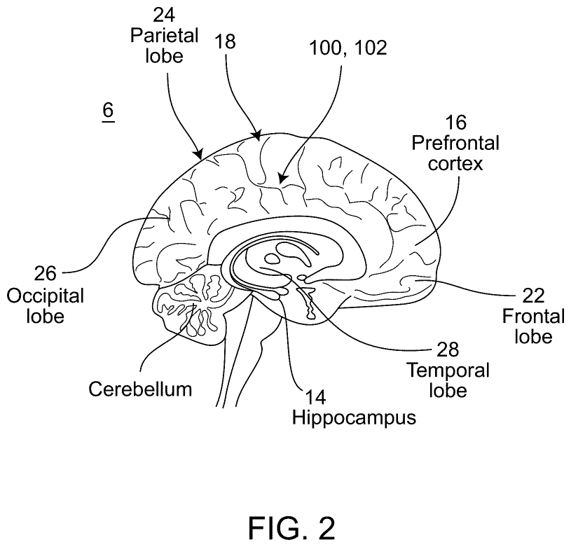

FIG. 2 is an illustration of areas of the brain 6 which operate to facilitate functions such as neurogenesis and angiogenesis. Neurogenesis is the process by which new neurons are formed in the brain 6. An increase in neurogenesis speeds up the recovery of memory capacity. Angiogenesis is the formation of new blood vessels. This process involves the migration, growth, and differentiation of endothelial cells, which line the inside wall of blood vessels. The process of angiogenesis is controlled by chemical signals in the body. FIG. 2 shows the hippocampus 14 and the prefrontal cortex 16 of the brain 10. Most cognitive effects are found in executive function, memory, and attention with increased brain volumes in hippocampus 14 and prefrontal cortex 16. The hippocampus 14 is a major component of the brain of humans and other vertebrates. Humans and other mammals have two hippocampi, one in each side of the brain. The hippocampus is part of the limbic system, and plays important roles in the consolidation of information from short-term memory to long-term memory, and in spatial memory that enables navigation. The prefrontal cortex 16 is the cerebral cortex 18 covering the front part of the frontal lobe 22. It is associated with executive function including planning complex cognitive behavior, personality expression, decision making, and moderating social behavior. Aerobic exercise increases hippocampal blood flow carrying more oxygen and nutrients, assisting in neurogenesis and angiogenesis.

An earlier model of guided plasticity improvement within the context of cognitive motor training depicted how the different modalities of cognitive-motor training, sequential or simultaneous, lead to increased neuroplasticity and improved cognitive performance.

Physical exercises trigger a sequence of neurophysiological mechanisms, which promote neuroplasticity but are transient and time-constrained and produce a facilitation effect.

Cognitive training is assumed to guide targeted neural networks and cognitive processes. This provides a guidance effect of cognitive exercises which have been correlated to distinct survival mechanisms of newborn cells.

The newly-generated cells created through exercise in the hippocampus 14 do not necessarily survive. More than half of them die within several weeks. Animal studies have shown that the survival rate of newly-generated cells is substantially increased in response to cognitive exercise.

A neural network 100 consists of many interconnected networks of neurons 102. In FIG. 2, the reference numerals 100 and 102 refer to the large number of neural networks and neurons distributed throughout the brain 6. In the brain 6 a typical neuron 102 collects signals from others through a host of fine structures called dendrites. The neuron 102 sends out spikes of electrical activity through an axon, the output and conducting structure, which can split into thousands of branches. A neural network 100 receives data at an input and provides a response.

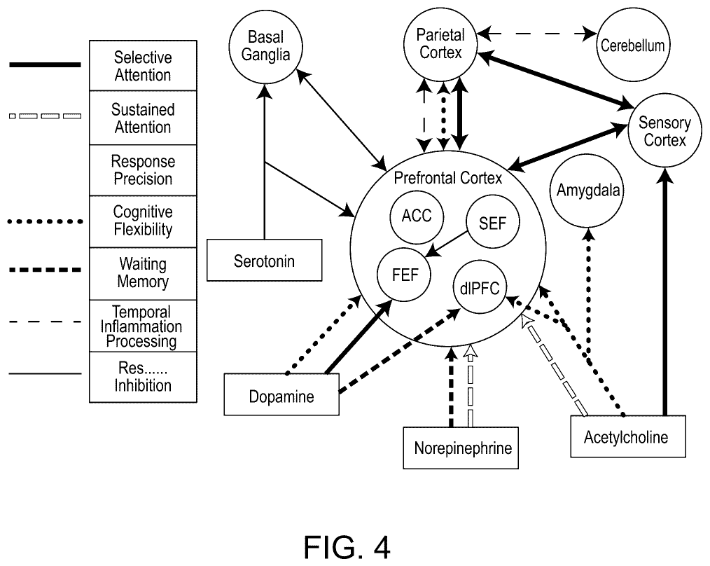

Each neural network 100 is associated with one condition. The present subject matter sets up stimuli to trigger a selected neural circuit (FIG. 4) and uses different neural circuits to improve cognitive abilities. This method trains cognitive abilities that have been disrupted. These abilities include memory, decision making, and processing of information. All stimuli or a predominant body of stimuli are aural. In one preferred form each sequence of steps comprises providing aural stimuli to the user.

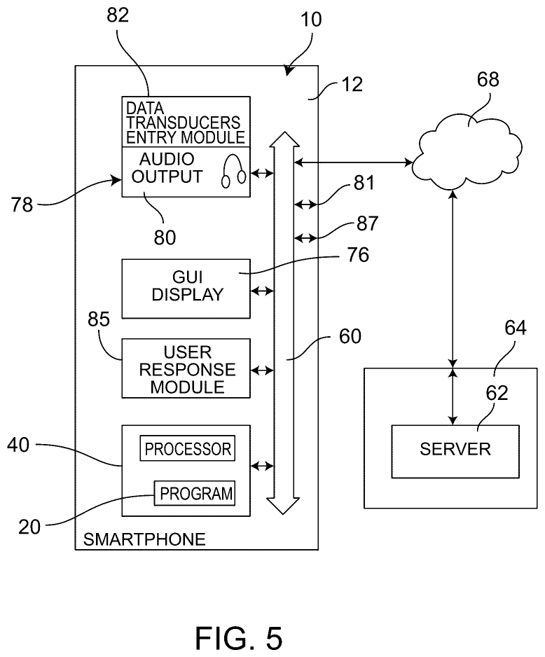

Visual or other stimuli may be used, but they provide a distraction from the activity that the user is focused on. Consequently, use of non-aural stimuli limits the scope of physical activities which may be used in conjunction with the cognitive training. Auditory stimuli interact with neural networks 100 associated with particular conditions, such as, ADHD, PTSD, brain fog, depression, and Parkinson's Disease. As a user performs exercise, the user 1 performs cognitive tasks in accordance with audible instructions from a processor. The user's vital signs are monitored by sensors, such as the heart rate monitor 48 (FIG. 1). The heart rate monitor 48 responds to a user's physical condition and provides an alarm via the said first interface 81 (FIG. 5) in response to a predetermined threshold.

Signals indicative of these vital signs and other information relating to the condition of the user's body are provided to a processor 40. The signals are processed to determine if the exercise is aerobic and may issue warnings to the user 1 regarding physical conditions. Simultaneously, the user 1 participates in a neuroscience-based cognitive training exercise program 20. The interface between the user 1 and the cognitive training exercise program 20 comprises the interactive device 10 which provides a stimulus to the user 1 and creates a signal when the user 1 responds to the stimulus. The interface may be the smartphone 12. The user 1 receives stimuli in the form of audio-based cognitive exercises and responds to the stimuli by making an entry into the keyboard 43 on the smartphone 12. The user 1 is instructed to perform the cognitive tasks requiring responses to stimuli in accordance with instructions. Performance of the user 1 in meeting requirements indicates cognitive ability from which increase or decrease in cognitive ability may be measured. The user provides physical interaction in response to action in the brain area selected by performance of an executed routine in the app.

One significant measure of performance is the time from production of the stimulus to the initiation of a response. A preselected time window is selected in which the user must respond in order to have a response registered. After the window closes, another stimulus is issued. A training mode may also be provided. In the training mode a next stimulus is not issued until after the user provides a response.

This system and method provides for efficiency in cognitive training. The user 1 will not have to invest time in a variety of time-consuming and non-measurable health-promoting activities. Significantly, the present subject matter comprises the first mobile-based simultaneous aerobic exercise and cognitive remediation program for mental health, neurocognitive, and neurodegenerative conditions. As stated earlier current available exercise and cognitive training equipment costs approximately $9000.00, which can be prohibitive. This system and method provides for efficiency in cognitive training. A user 1 will not have to invest time in a variety of time-consuming and non-measurable health-promoting activities.

Exercise requiring greater physical motor skill may lead to increased recruitment of frontal-parietal cognitive control circuitry engaged in executive and attentional processing. Aerobic exercise may engage broader changes in cognitive and motor circuitry corresponding to changes in the hippocampus 14 and the cerebellum 18.

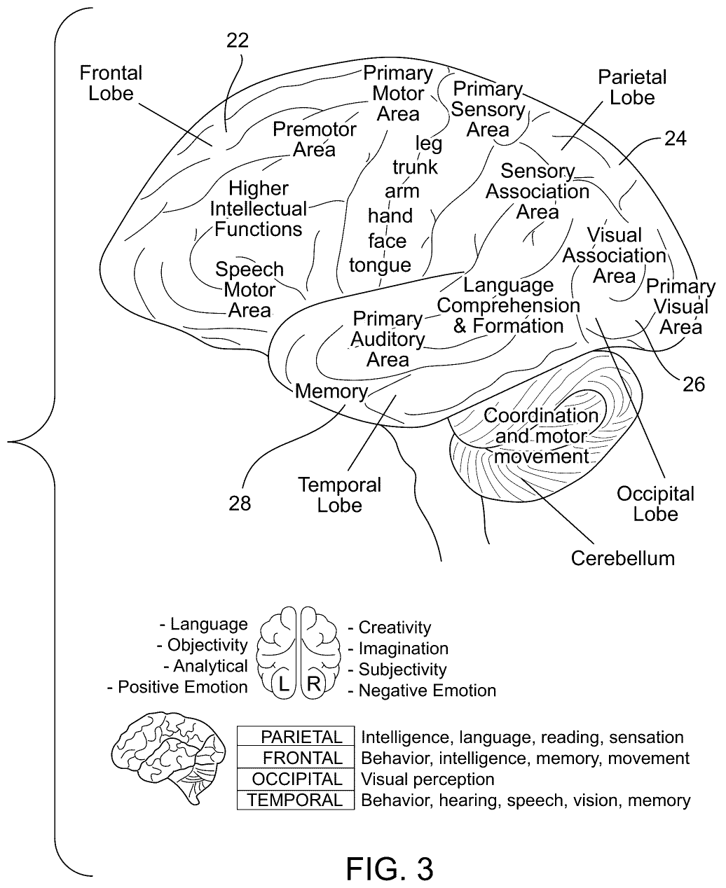

FIG. 3 is an illustration of various lobes in the brain and cognitive skills associated with the lobes. The frontal lobe 22 is associated with speech motor area, higher intellectual functions, premotor area, and primary motor area. Specific example of these functions are behavior, intelligence, memory, and movement. A parietal lobe 24 is associated with primary sensory area and sensory association area. Examples of these functions include intelligence, language, reading, and sensation. An occipital lobe 26 is associated with visual association area and primary visual area. Examples of functions controlled by this lobe include vision and love. A temporal lobe 28 is associated with memory, primary auditory area, and language comprehension and formation. Functions controlled by this lobe include behavior, hearing, speech, vision, and memory. Actual activation of brain areas in MRIs can confirm neural activity in regions and networks in the brain corresponding to selected cognitive functions.

FIG. 4 is a diagram of neural circuits underlying cognitive domains. FIG. 4 describes the relationship between ADHD-related functional cognitive domains and the neural circuits that steer them. The circuits are governed by neuromodulators, which are substances that can change the effect on a nerve of other neurons. Neuromodulators can control the amounts of neurotransmitter released in response to other stimuli. This diagram shows the interaction of neuromodulators and brain regions important for specific functional domains. It shows neuromodulatory input to brain structures implicated in one or more functional domains as well as some of the major connections. The neuromodulators act upon neurotransmitters: serotonin, dopamine, norepinephrine and acetylcholine are primarily released by specific subcortical nuclei. Serotonergic neurons are located in the dorsal raphe nuclei, dopamine neurons that project to prefrontal cortex in the ventral tegmental area, norepinephrine-releasing neurons in the locus coeruleus, and cholinergic neurons in the nucleus basalis. Other portions of the brain in FIG. 4 include the basal ganglia, parietal cortex, cerebellum, sensory cortex, and amygdala. Areas in the prefrontal cortex are the anterior cingulate (ACC), the supplementary eye field (SEF), the dorsolateral prefrontal cortex (dlPFC), and the frontal eye field (FEF). Regions including the prefrontal cortex, the cerebellum, and basal ganglia are suggested to be involved in neuromodulator systems. A core component of cognitive control is response inhibition, which is facilitated by the prefrontal cortex and the ACC. Recent studies tend to connect ADHD as a disorder of executive functioning and cognitive control. Studies show that targeted cognitive training improves ADHD symptoms. Training individuals that have ADHD in a specific domain, such as working memory, has resulted in some degree of efficacy. The present subject matter presents an adjunctive treatment for ADHD.

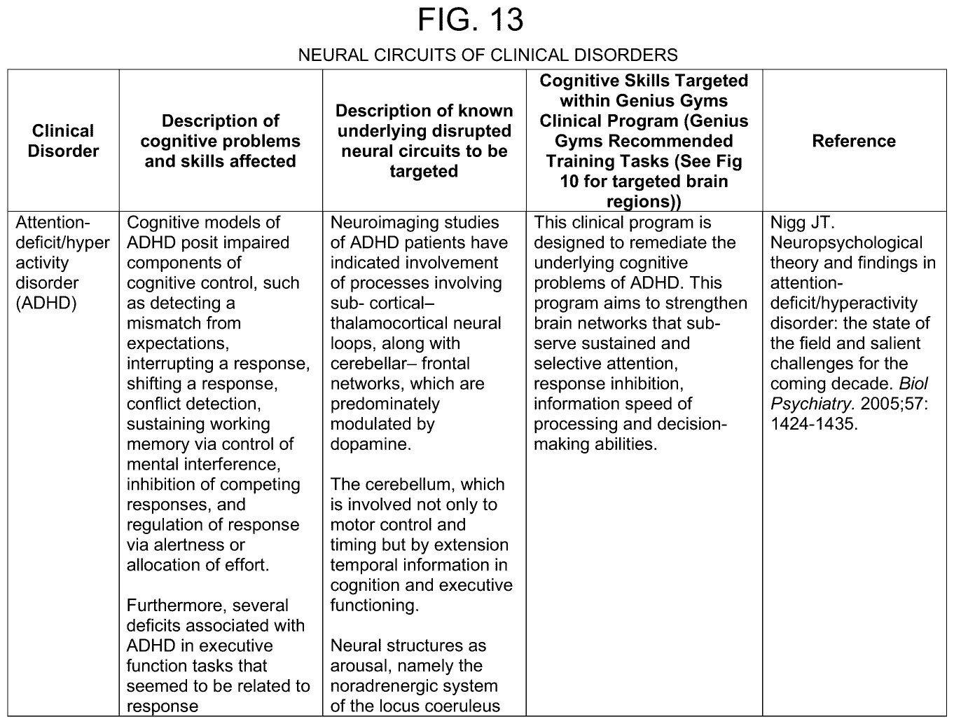

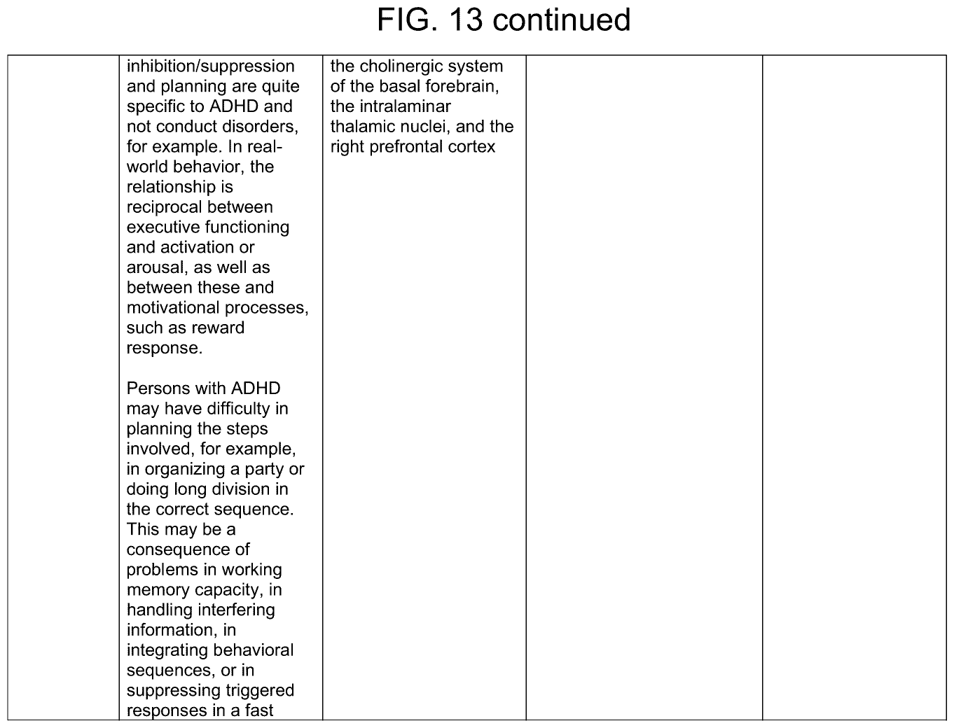

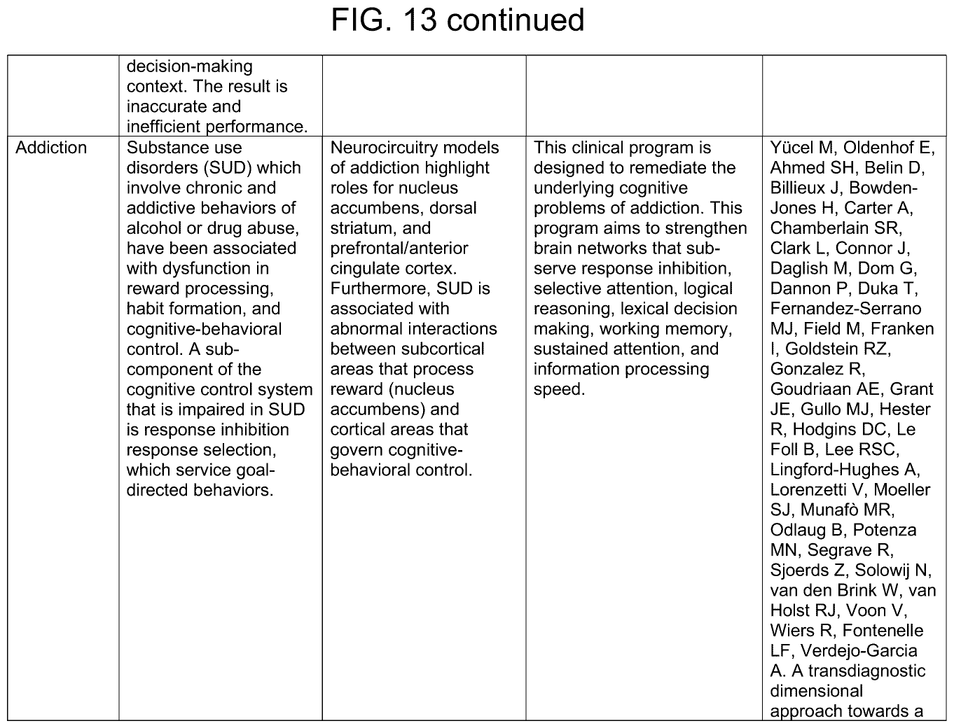

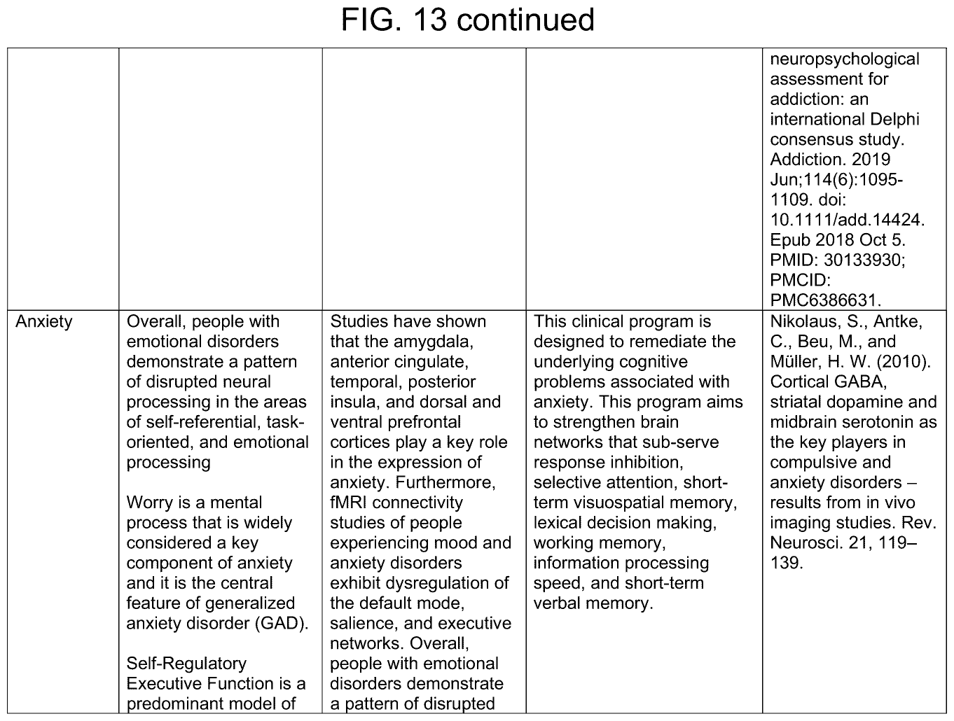

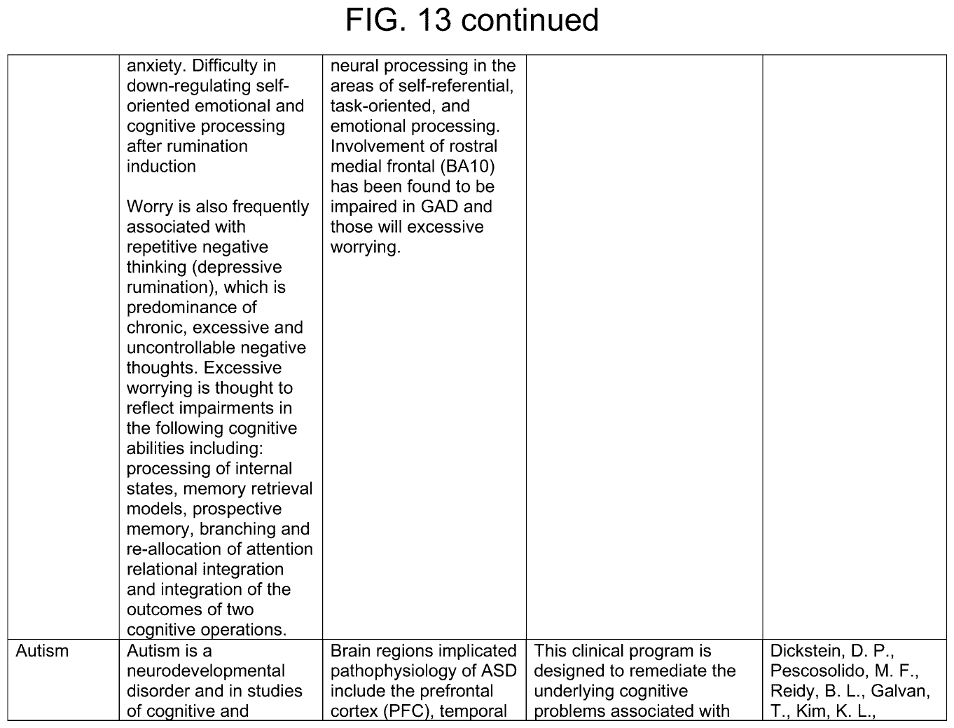

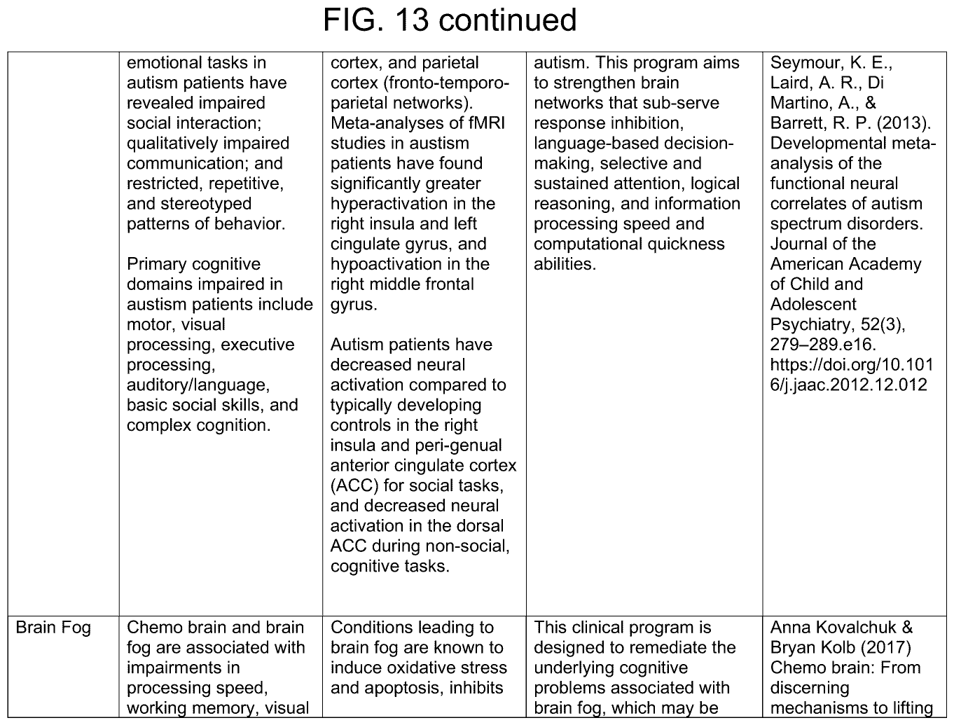

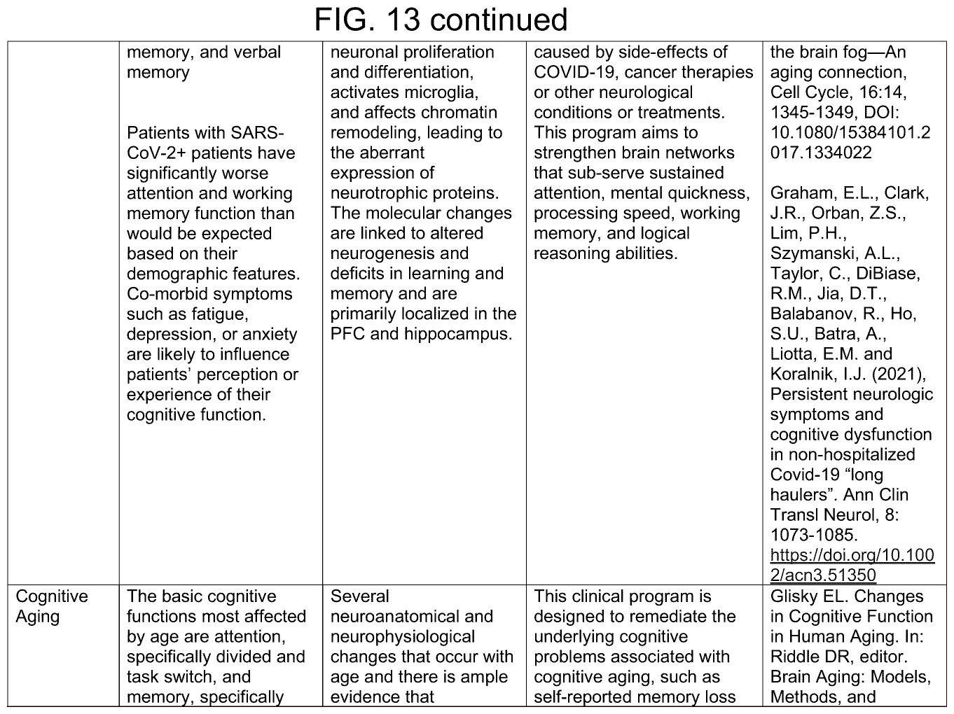

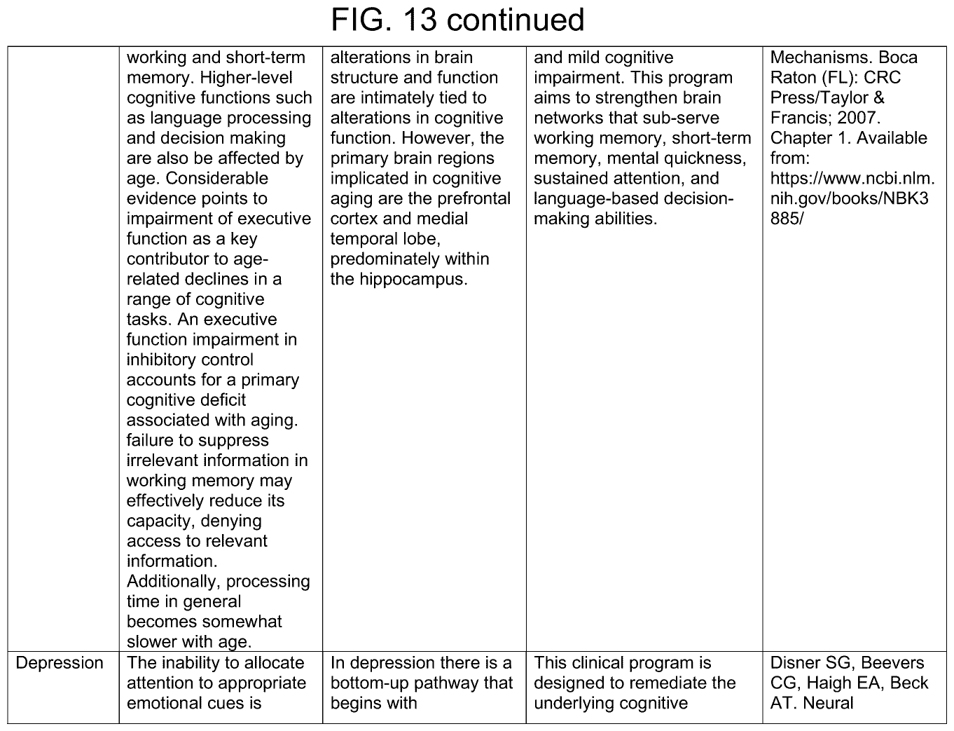

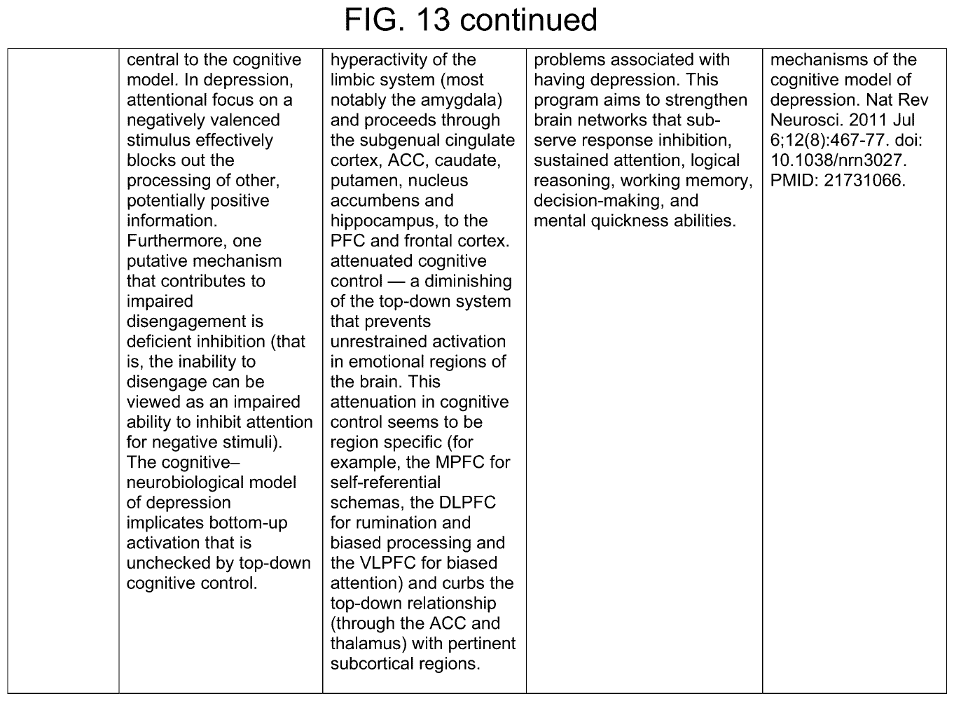

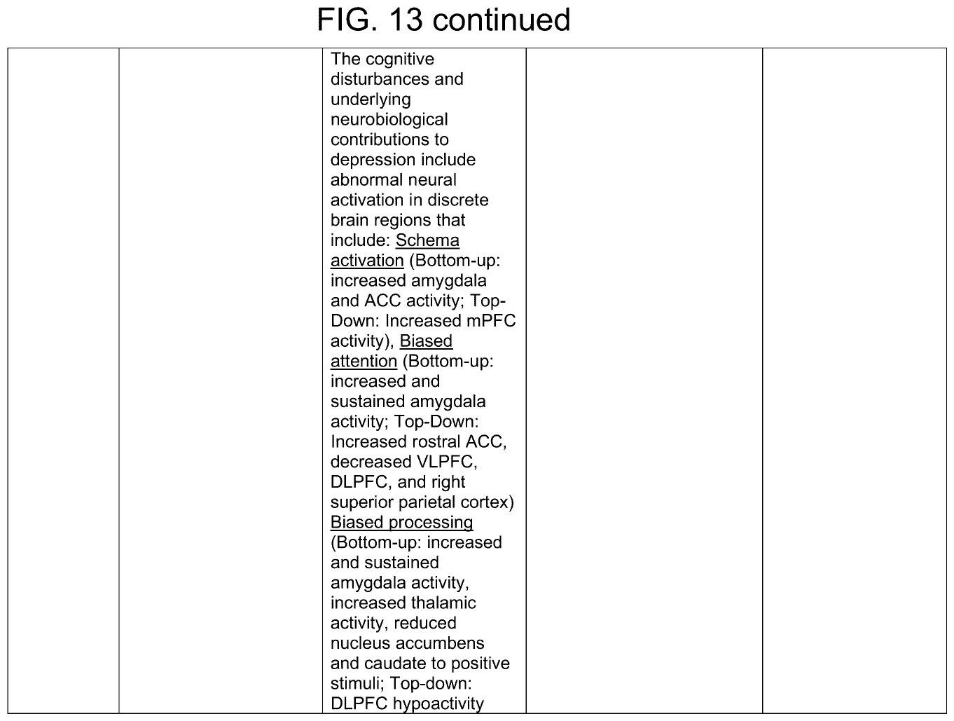

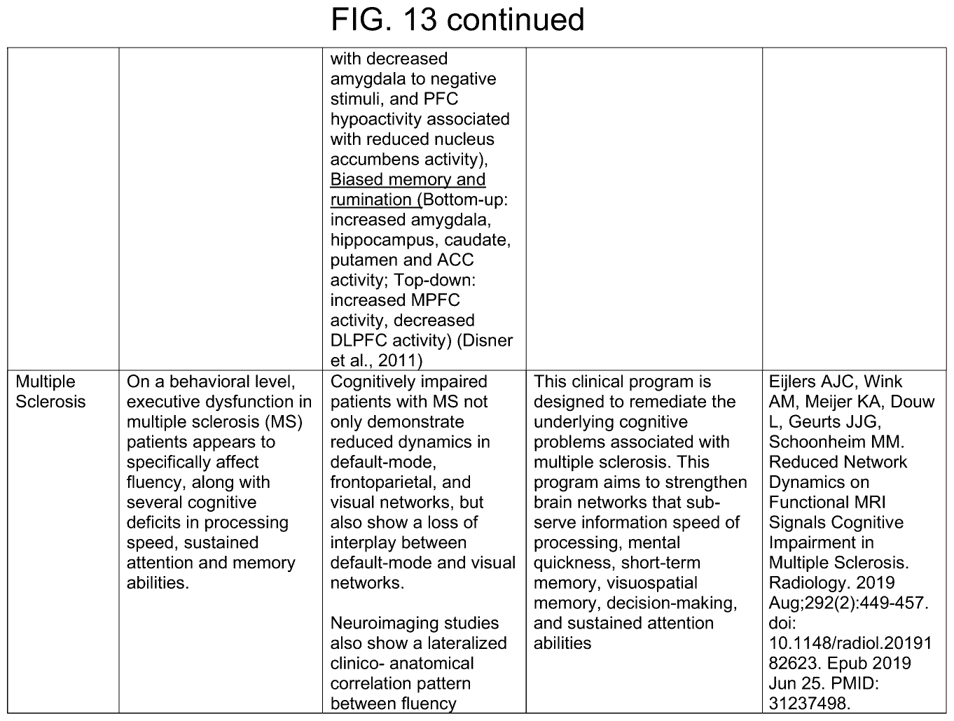

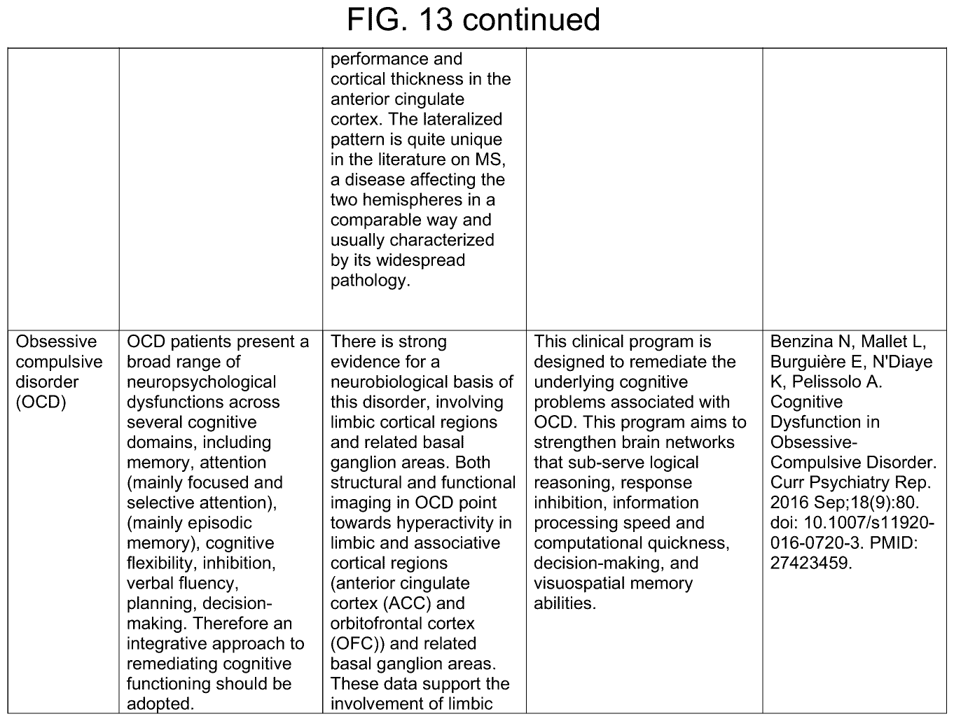

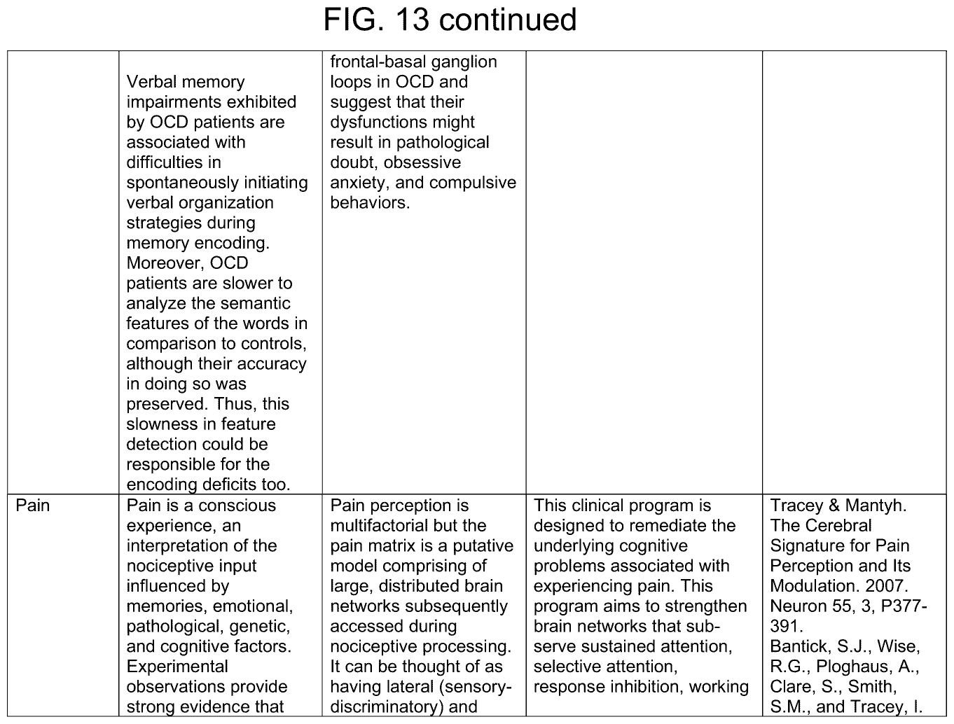

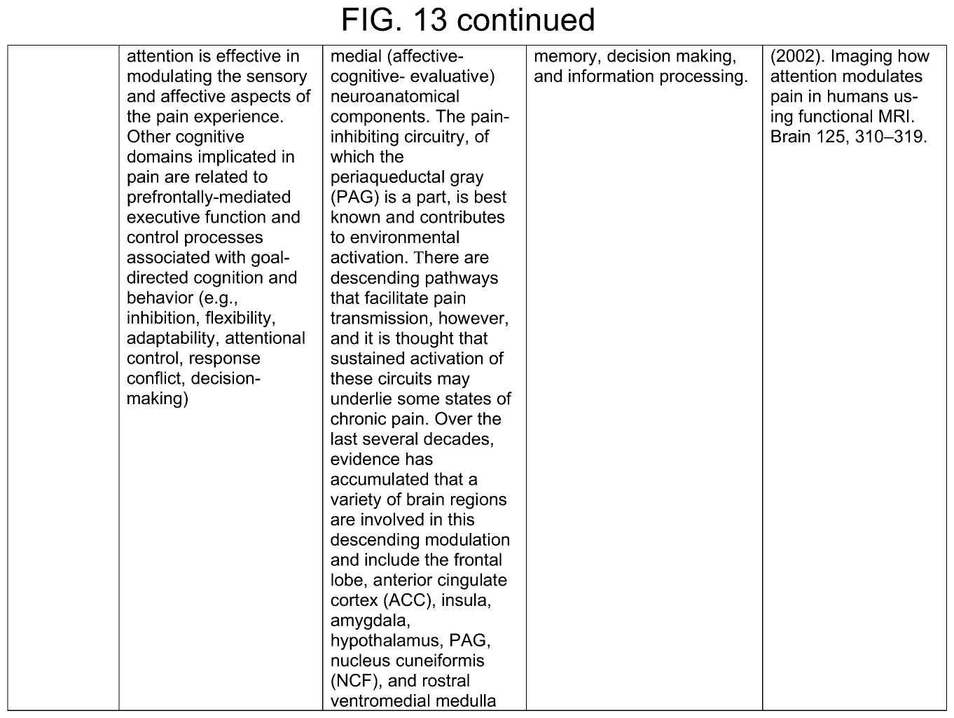

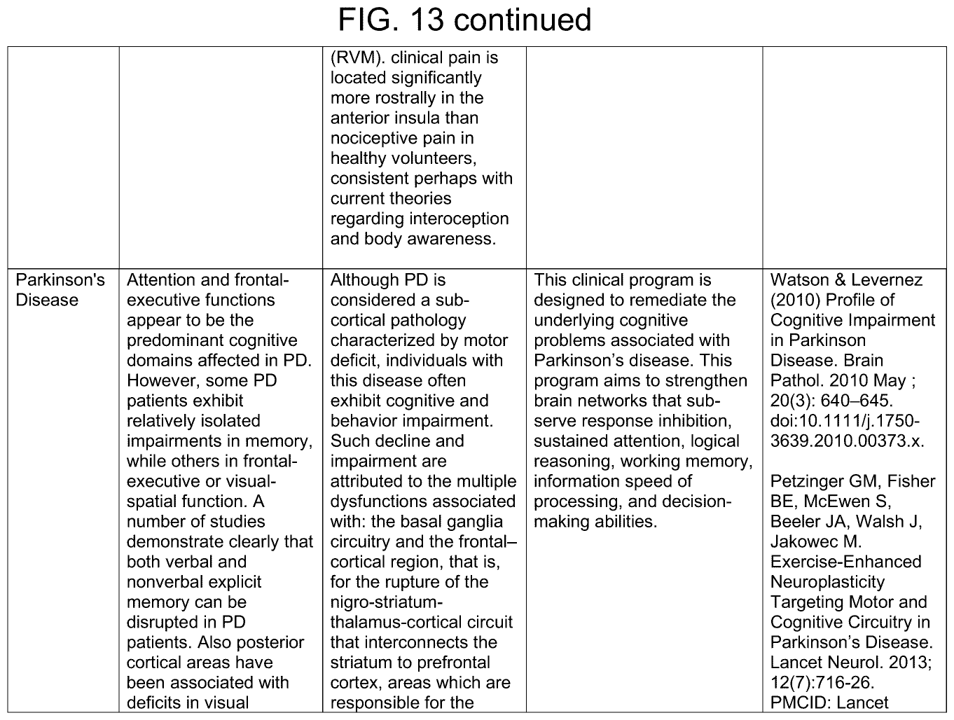

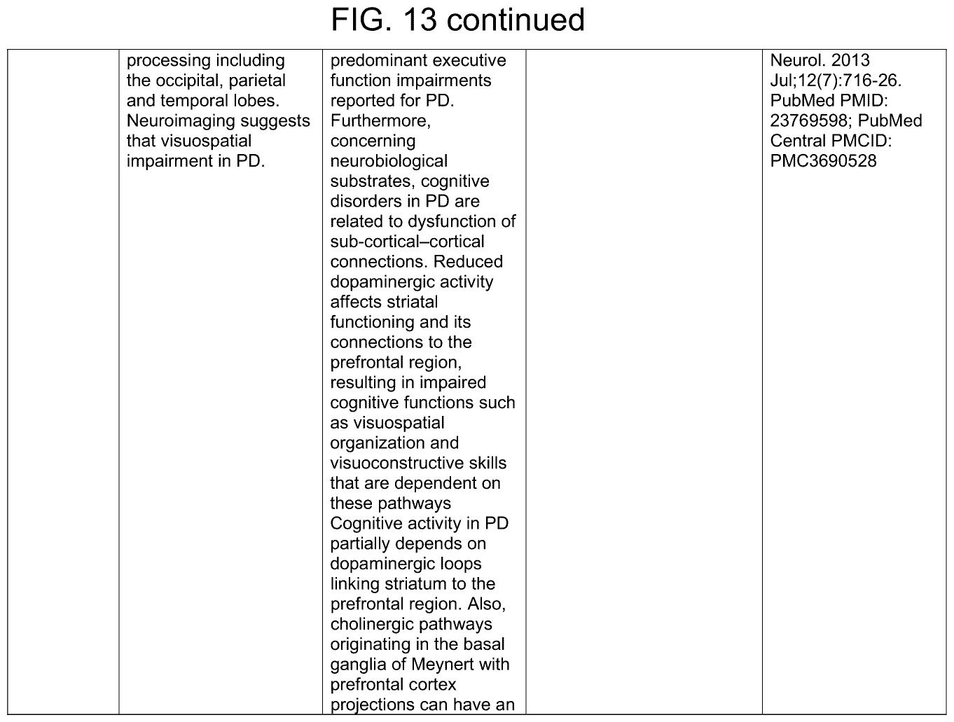

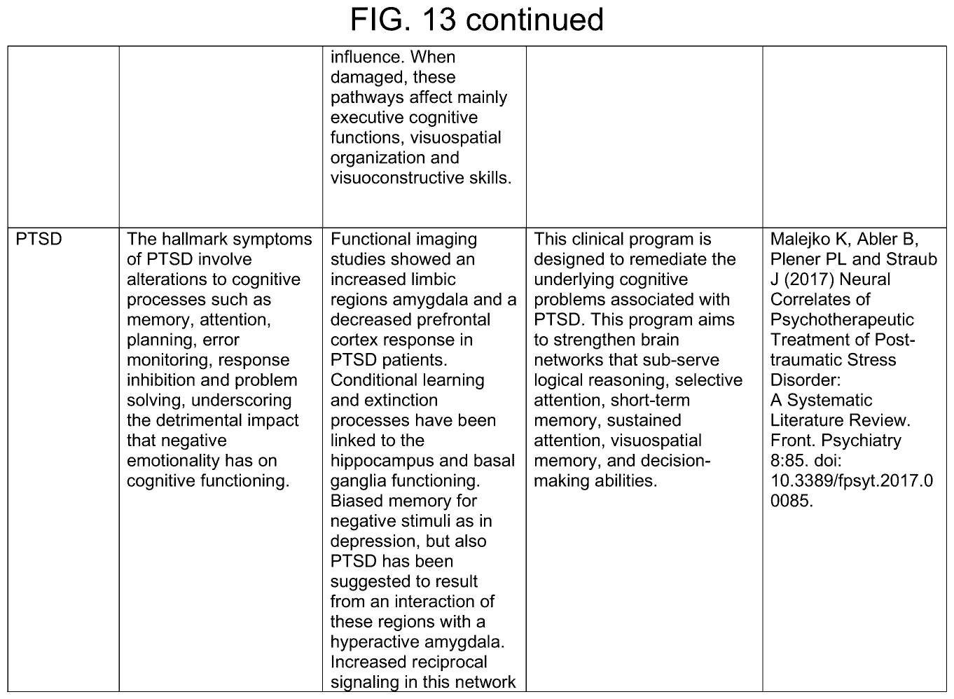

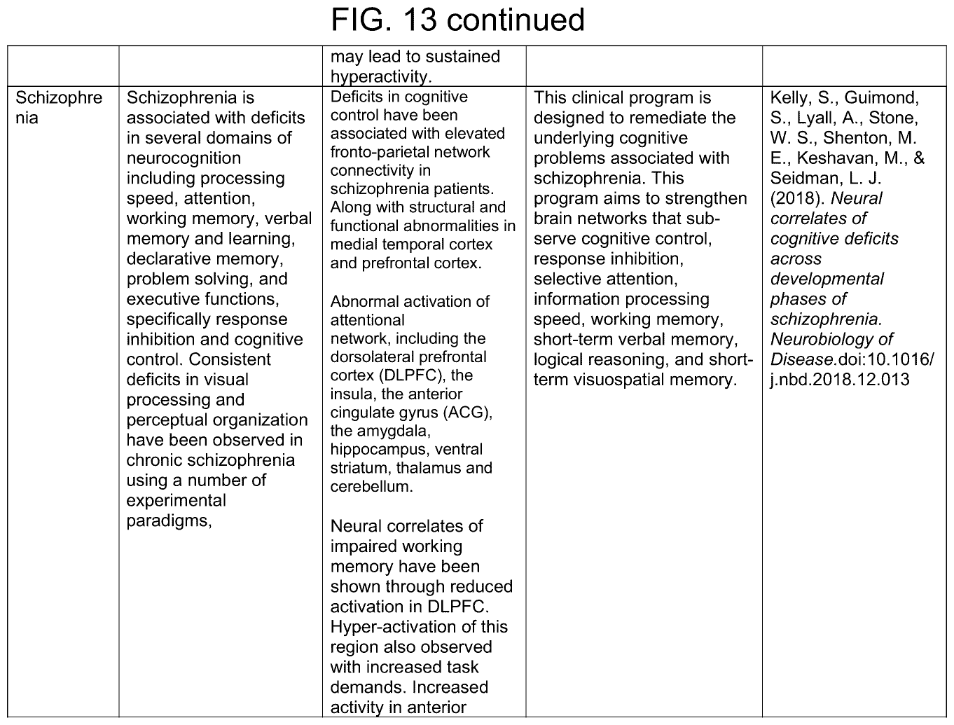

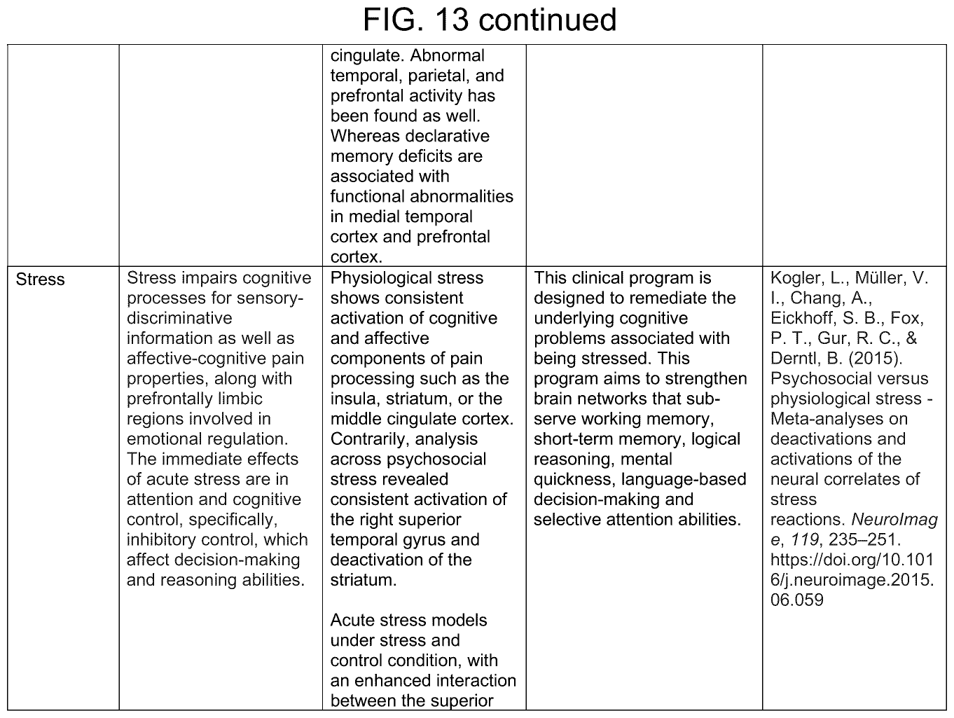

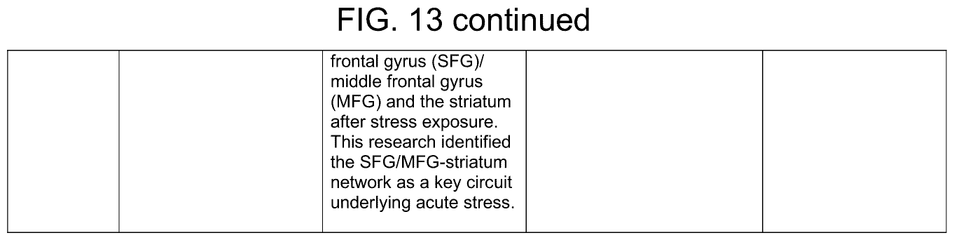

Studies seem to identify that ADHD with distinguishable symptoms are caused by deficits in either the modulation of cognitive control and attention neural networks. FIG. 13 describes several clinical disorders and provides details of the disrupted neural circuits and cognitive skills exercises in that disorder and then describes targeted routines to repair these disrupted circuits and cognitive skills. For example, neural circuits impaired in ADHD involve processes involving subcortical thalamocortical neural loops, along with cerebellar frontal networks, which are predominately modulated by dopamine leading to impairments in primarily cognitive control, attention, speed of processing and decision-making cause which are targeted with the clinical routine to address this deficits.

Certain neural networks are more impaired in particular neurocognitive or neuropsychiatric conditions. The present subject matter sets up stimuli to trigger a selected neural circuit and uses different neural circuits to improve cognitive abilities. This method trains cognitive abilities that have been disrupted. These abilities include memory, decision making, processing of information. All stimuli or a predominant body of stimuli are aural. Visual or other stimuli may be used, but they provide a distraction from the activity that the user 1 is focused on. Consequently, use of non-aural stimuli limits the scope of physical activities which may be used in conjunction with the cognitive training. Auditory stimuli interact with neural networks associated with particular conditions, such as, ADHD, PTSD, brain fog, depression, and Parkinson's Disease. As a user performs exercise, the user performs cognitive tasks in accordance with audible instructions from a processor. The user's vital signs are monitored by sensors. Signals indicative of these vital signs and other information relating to the condition of the user's body are provided to a processor. The signals are processed to determine if the exercise is aerobic and may issue warnings to the user 1 regarding physical conditions. Simultaneously, the user 1 participates in a neuroscience-based cognitive training exercise program. The interface between the user 1 and the cognitive training exercise program comprises a device which provides a stimulus to a user 1 and creates a signal when the user 1 responds to the stimulus. The interface may be the smartphone 12 (FIG. 1). A user 1 receives stimuli in the form of audio-based cognitive exercises and responds to the stimuli by making an entry into the keyboard 43 on the smartphone 12. The user 1 is instructed to perform the cognitive tasks requiring responses to stimuli in accordance with instructions. Performance of the user 1 in meeting requirements indicates cognitive ability from which increase or decrease in cognitive ability may be measured.

The present system combines physical exercise with cognitive tasks with audio stimuli. These are presented to a user 1 who is performing physical exercise, preferably aerobic exercise. The present method and system stimulate individual neural circuits to improve cognitive abilities. PTSD, ADHD, and other conditions may be separately addressed. Combined neurocognitive exercise has been found to activate underlying known neural networks. Separate neural networks are associated with corresponding conditions that each affect various cognitive abilities, lending to a transdiagnostic application. Cognitive rehabilitation training used neuroscience-based cognitive tasks in simultaneous conjunction with performing aerobic exercise with the goal of assistance in cognitive rehabilitation through promoting physical exercise with targeted dual tasks.

Each task in this system trains different cognitive processes. All of the exercises include cognitive training elements of psychomotor response training, working memory training, cognitive motor dual task training, and sustained attention training. In psychomotor response training the time between receiving an audio stimulus and performing a physical response is measured. In working memory training, each user 1 needs to hold the instructions to the task in their working memory to be able to do the task appropriately. In cognitive-motor dual task training, all tasks need to be completed while simultaneously engaging in aerobic training. In sustained attention training, a user 1 needs to keep a sustained level of concentration to actively and accurately respond to every stimuli the user 1 hears. Attention is the gate through which sensory information enters our conscious experiences and is therefore a critical cognitive ability which needs to be continuously tuned and refined.

Most mental health, neurocognitive and neurodegenerative conditions, such as depression, ADHD, and Parkinson's disease have disrupted underlying brain networks leading to increased cognitive and behavioral impairments and diagnostic presentation of the condition. This program is designed to engage and train specific neural circuits of brain dysfunction that are central to cognitive deficits associated with specific neuropsychiatric and neurodegenerative conditions. Currently, most major psychiatric conditions are treated with pharmacological interventions, such as ADHD, designed to treat the symptoms of the disease. For example motor deficits in Parkinson's disease is treated with pharmacological interventions, but they are unable to treat the known cognitive disturbances in that particular condition by systematically targeting neurobiological substrates.

FIG. 5 a block diagram of an apparatus for improving, maintaining, or remediating impairments in selected cognitive skills. It is a system used in performance of the current method. The apparatus uses the portable interactive device, the portable interactive device comprises a first interface 81 providing stimuli to a user and a second interface 83 receiving inputs from the user. The second interface 83 couples to a response module 85. The heart rate monitor 48 (FIG. 1) responds to a user's physical condition and may provide an alarm via the first interface 81 in response to a predetermined threshold. The app 30 operates in the portable interactive device. The training program 20 comprises exercise routines each associated with a cognitive condition and each routine comprises a sequence of cognitive exercises, each cognitive exercise being associated with at least one cognitive skill. The program 20 provides instructions to the user for execution of cognitive exercises, provides stimuli for a preselected number of repetitions, and receives inputs for the user 1 in response to stimuli. The program 20 comprises a suite of routines each for engaging neural circuits associated with selected cognitive conditions, whereby a mobile system for simultaneous cognitive remediation and physical aerobic exercise is provided. Each routine comprises a sequence of selected cognitive exercises. The cognitive exercises are selected to address the most significant cognitive function for the selected cognitive condition being addressed.

The apparatus 8, an interactive device 10, may take many forms. In each form the apparatus 8 will take a physical form with which the user 1 may interact. In another form, the interactive device 10 may comprise a console in a stationary exercise apparatus. A transducer module 78 comprises first and second transducers 80 and 82 which present stimuli to a user 1 and receives responses. The first transducer 80 may be included in the headphones 50 or ear buds worn by the user 1 (FIG. 1). The first transducer 80 may be connected by a wire to the smartphone 12. Alternatively, in a wireless embodiment, the first transducer 80 is coupled to the smartphone 12 by the Bluetooth circuit 54. The second transducer 82 may be included in the touch input unit 44 (FIG. 1) or the data input 42 in the smartphone 12. A graphical user interface (GUI) 76, such as an LED display, facilitates communication between the user 1 and the program 20 and a processor 40 executes a program to inform the user 1 of instructions and stimuli and process responses of the user 1 in accordance with the instructions of the cognitive exercise. In further preferred forms, the interactive device 10 will communicate with a remote facility at which further processing is done. Further processing may include construction of statistical libraries and performing programs 20 which make use of statistical data to customize programs for individual users 1 or groups of users 1.

The transducer module 78, the GUI 76, and the processor 40 are interconnected by a data bus 60. Local processing is done in the processor 40. However, the smartphone 12 may be coupled via the data bus 60 to a remote server 62 at a remote location 64. The data bus 60 is a third interface 83 to couple data to the server 62 to provide data to the remote server and to receive processed data.

The smartphone 12 and the server 62 communicate via the Cloud 68. Information may be sent to the server 62 to keep historical records for the user 1. Additional processing may be performed at the server 62 to generate additional statistical functions based on the history of the user 1. Also, data from a virtually unlimited number of other users may be transmitted to the server 62. Individual statistics are generated for each of the other users. Composite data collections may be formed to provide data sorted by various descriptions. Statistics for selected populations may be generated. Data mining may be performed to generate new knowledge.

The program 20 is housed in the processor 40. The app 30 may be provided to be loaded in the processor 40 via cell phone connection or Wi-Fi. It could be embodied in an SD memory card plugged into the smartphone 12 or may be embodied in an app 30 from the iPhone App store or from the Google Play store. Effectiveness of the exercises is correlated with the exercises that are performed in order to optimize improvement in selected cognitive areas with respect to the amount of exercise needed.

In one preferred form the interactive device 10 is portable. The user 1 is not constrained to exercise at a fixed location such as a stationary bicycle. Therefore, the user 1 may engage in exercises such as walking, cycling, running or sport-specific. The range of physical exercises is virtually unlimited.

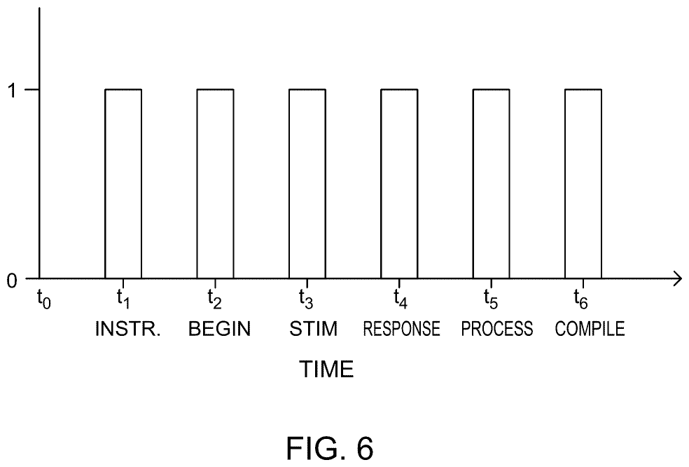

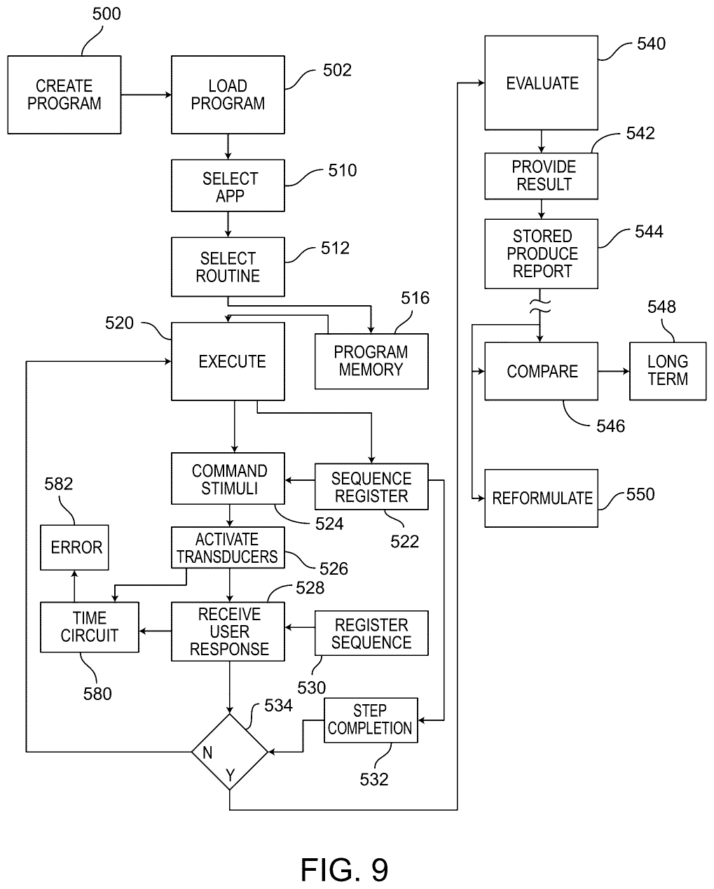

FIG. 6 is a timing chart showing the sequence of events in a nominal cognitive exercise in which the abscissa is time. FIG. 6 illustrates the interaction between the user 1 and the system. Different exercises have their own sequences of events. These different sequences will be accomplished in the manner illustrated in FIG. 6. The method of the present disclosure comprises the steps illustrated in FIG. 9. The ordinate indicates the occurrence of a labeled event. The “zero level” indicates the absence of a step. A transition from the zero level to the “one level” indicates that a step is beginning. The value returns to zero at the end of the step. The width of each step is not to scale. Duration of each step may vary in comparison to another step in different exercises. The steps of the exercise occur in a sequence at times t₁, t₂, t₃, . . . . In each row below the time of occurrence is listed, the name of the step is provided, and comments follow. The method begins at time t₀.

| | | |
|---|---|---|
| t₀ | Initialize | The user gets in position to perform the physical exercise and |
| | | prepares to use the interface device. |
| t₁ | Instructions | The processor tells the user what the cognitive exercise comprises |
| | | and how the user is to respond to stimuli. The processor may |
| | | identify stimuli that will be provided. The user will be instructed |
| | | to take a first action in response to a first stimulus and to take a |
| | | different action in response to a differently defined stimulus. |
| t₂ | Begin | The user indicates readiness to perform. |
| t₃ | Stimulate | The program provides a stimulus. For example, the stimulus could |
| | | be a letter or numeral or word. In an exercise related to reaction |
| | | time, the stimulus may comprise a beep. |
| t₄ | Respond | The user takes the action to inform the program of the response to |
| | | the stimulus. |
| t₅ | Process | The signal produced indicative of the response is sent to the |
| | | processor 40 and operated upon in accordance with the algorithm |
| | | in the program 20 characterizing the exercise to provide a measure |
| | | of user performance. |
| t₆ | Compile | This step may be removed in time from the other steps. Data may |
| | | be aggregated from any number of tests and grouped in |
| | | accordance with classifiers to develop data sets for providing |
| | | statistics. |

The steps at t₃ and t₄ may repeat in a cycle. The number of cycles is equal to the number of stimuli and responses in a sequence defining an exercise. The compile step is optional. However it is extremely important when operators of the method are seeking to individualize a set of exercises to provide the best results for a user. This information is compared to subsequent information to monitor changes in cognitive abilities of the user 1 in response to performance of the method. A scientist can interpret results in order to individualize and customize the program to provide the best results for an individual user. This method in one preferred form comprises the step of correlating the user's results to the cognitive exercises performed and performing a closed loop error correction process to establish efficacy in cognitive improvement.

The exercise program used to address a particular cognitive impairment comprises a number of routines. Each routine directed to a specific condition consists of a plurality of exercises with the selected exercises and repetitions being selected in accordance with the specific condition. The exercises each consist of a number of steps. Steps include such actions as providing a stimulus from the processor 40 and having the user 1 produce a response. The nature of exercises is discussed with respect to FIG. 12. In each exercise the techniques of FIG. 6 are used to implement each step in an exercise.

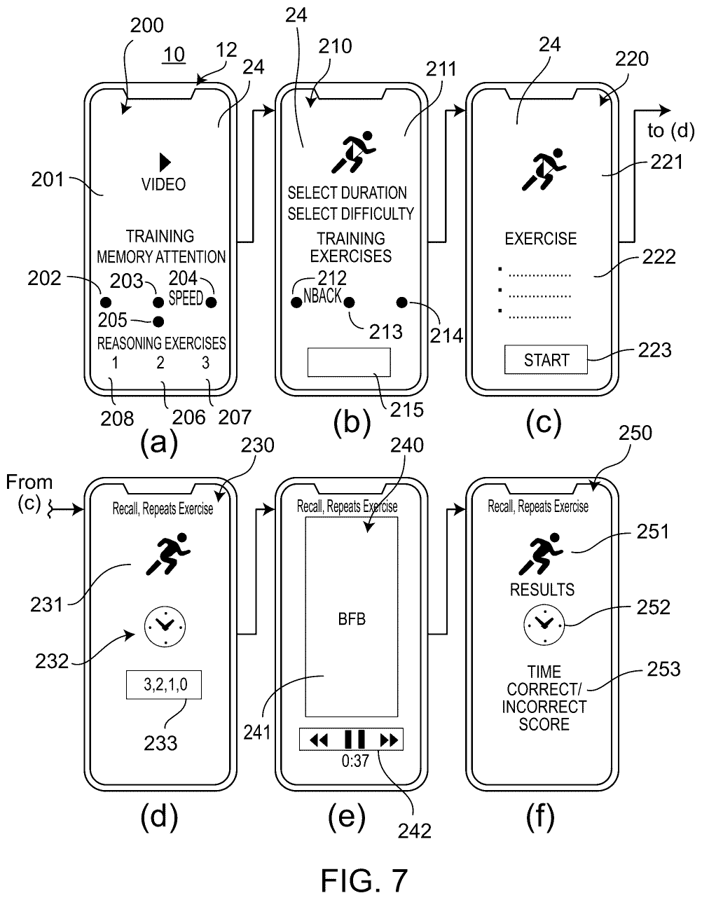

FIG. 7 illustrates content delivery to a user 1 performing an exercise. In this illustration the interactive device 10 is the smartphone 12. In one nominal application the program memory 40 provides successive sets of information, referred to as screens 200, 210, 220, 230, 240, and 250. The display 24 advises, informs, and instructs the user 1. The six successive screens 200 through 250 are illustrated in panels “a” through “f” in FIG. 7, respectively. Each screen has a line showing a selection on a first field leading to an initial display 24 on a next screen. In panel “a” the display 24 shows the user 1 available categories of training and exercises. The screen 200 comprises fields 201 through 207. Field 201 is an area displaying a video, which can be viewed by the user 1. The video may provide instructional media including still images, moving images, and audio. Fields 202 through 205 are assigned to routines for cognitive skills that include memory, attention, speed, and reasoning, respectively. The user 1 may slide the fields 202 through 204 to the left to reveal further training categories. Fields 206 through 208 illustrate fields for neuro training exercises which will become available when one of the fields, such as field 202, is selected. These fields each correspond to a type of exercise. In this illustration they respectively correspond to exercises entitled N-Back, Corsi Task, and Letter Memory Test. Each named exercise contains a preselected sequence of steps. If the user 1 selects the memory field 202, operation proceeds to a screen 210 in panel “b.”

The screen 210 comprises fields 211 through 215. Field 211 displays an image. Fields 212, 213, and 214 represent the same selections as fields 205, 206, and 207. These fields denote particular training exercises. Field 215 may be selected, giving the user 1 the option to randomize exercises. In field 212, N-Back is selected, and operation proceeds to screen 220 shown in panel “c.” Screen 220 includes fields 221, 222, and 223. Field 221 is an image giving the user 1 the option to select running of a video. Field 222 comprises a description of the exercise the user has selected, and field 223 is activated to begin the exercise. Starting the exercise by selecting field 223 moves operation to screen 230 in panel “d.” Screen 230 comprises fields 231, 232, and 233. Field 231 is an icon representing a user 1. Field 232 is a clock, and field 233 is a countdown timer leading to the beginning of the exercise.

When the countdown timer 233 reaches 0, operation proceeds to screen 240 in panel “e.” Screen 240 includes fields 241 add 242. Field 241 represents that the current program is proceeding. Field 242 allows the user 1 to pause, fast forward, or rewind the program. When the program winds down, the end of an exercise is indicated and the operation proceeds to screen 250 in panel “f.” Screen 250 comprises fields 251 through 253. Field 251 includes an icon and a title, “results.” Field 252 represents a parameter useful to the user 1 at the end of the exercise. In the present illustration field 252 comprises a clock display indicating current time. Field 253 is a report to the user 1 on results of the test. In the current illustration, the report describes a test having a duration of 3 minutes, with the user 1 having made 9 correct responses out of 10 questions for a score of 90%. A label is also provided as to rated skill level of the test. Levels are referred to as easy, intermediate, or genius level. These levels are defined with respect to FIG. 12.

In one performed form a routine is customized for a user in terms of duration, difficulty, particular instructions, and times allowed between stimulus and response. The customization is performed in response to correlation of the user's results based on performance in accordance with selected parameters.

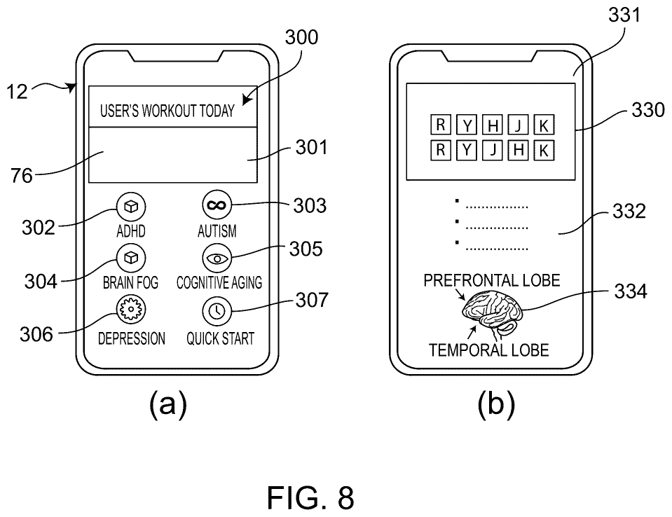

FIG. 8 is a graphical user interface 76 through which the user 1 cooperates with the program 20. FIG. 8 has parts “a” and “b” which comprise a sequence of two views of a graphical user interface through which the user 1 cooperates with the program 20. The graphical user interface 76 provides a succession of screens. A first screen 300 permits selection of a cognitive condition and election of the cognitive condition initiates a second screen 331. The second screen 331 describes a first cognitive skill related to the cognitive condition and provides instructions to the user for performance of a neural circuits exercise.

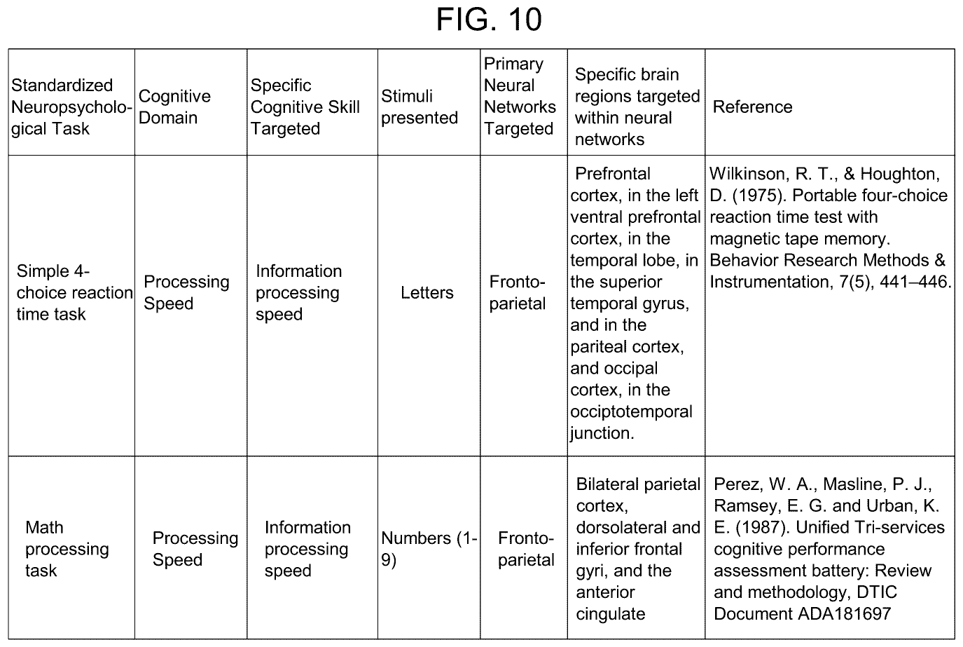

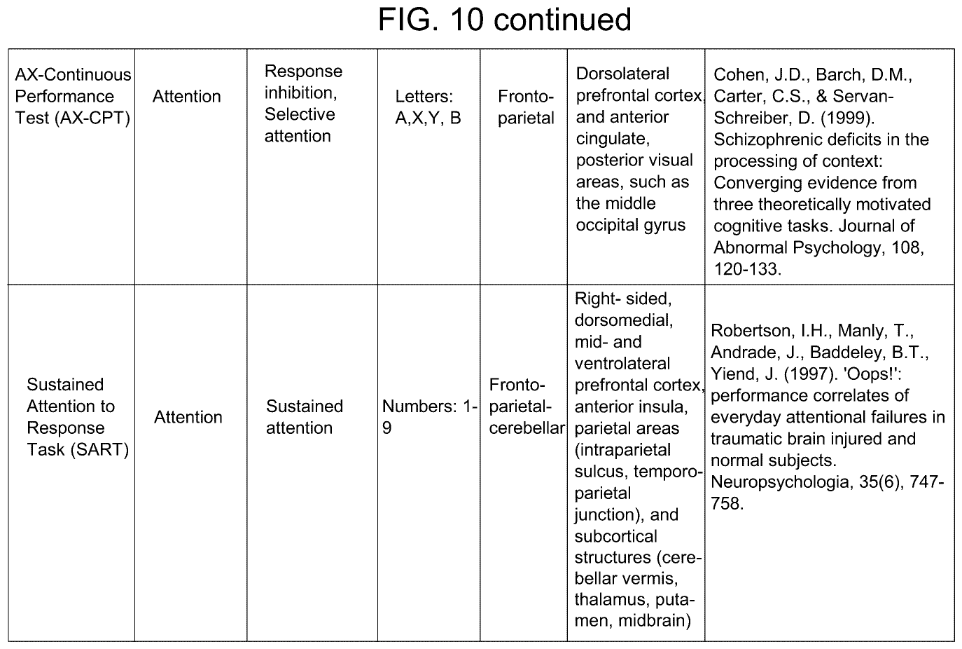

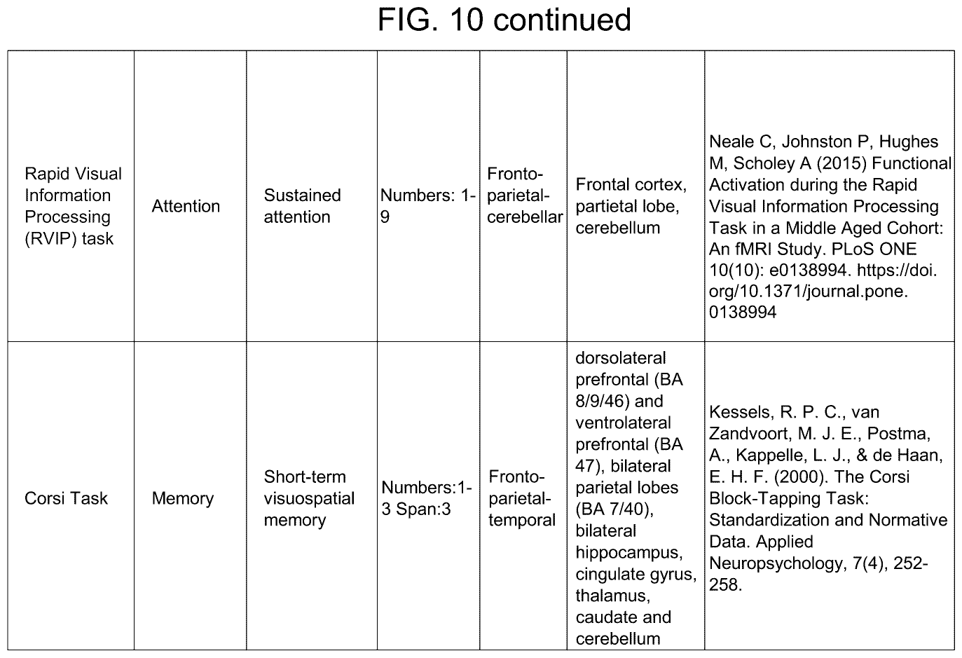

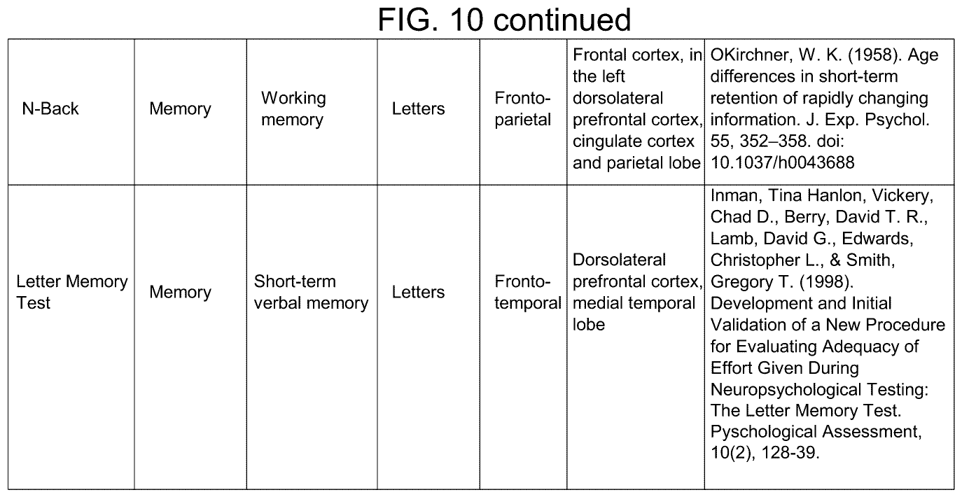

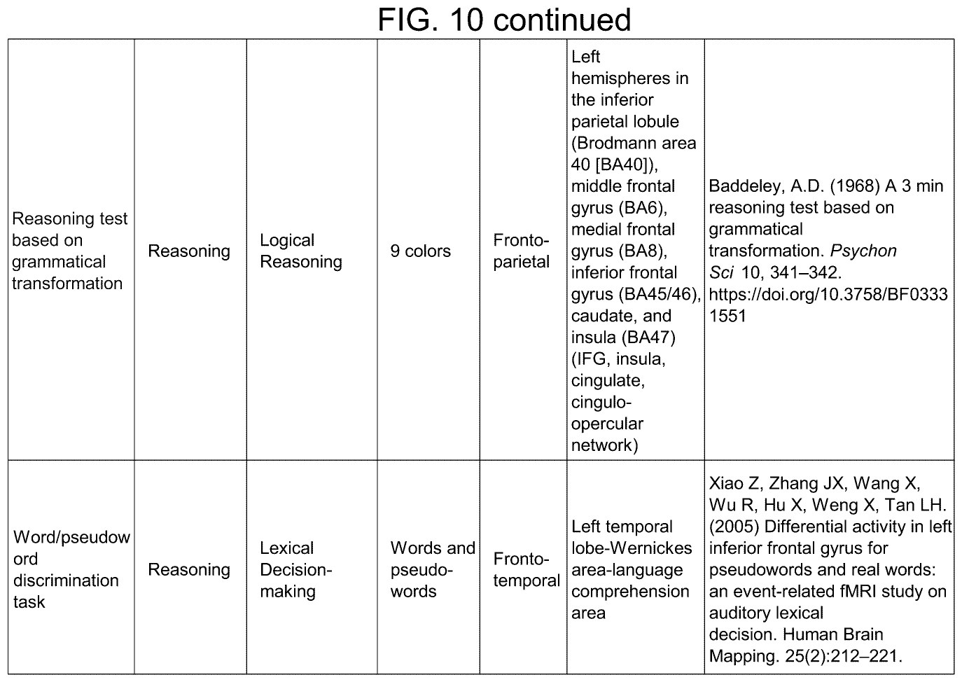

The smartphone 12 comprises a display 24 which shows a screen 300. In part “a” the screen 300 comprises fields 301 through 307. Field 301 shows text which may include the user 1's name and a statement of how to use the smartphone 12 to interface with the routine. In the present illustration the duration of exercises and an instruction to use headphones 50 are presented to the user 1. Fields 302 through 306 each allow selection of a clinical program associated with a particular mental health of neurocognitive condition. The exercises associated with a program are illustrated in FIG. 10. In FIG. 8 the fields 302 through 306 are associated with the programs for ADHD, Autism, Brain Fog, Cognitive Aging, Depression, respectively. ADHD aims to strengthen brain networks that subserve sustained and selective attention, response inhibition, information speed of processing and decision-making abilities. Depression aims to strengthen brain networks that subserve response inhibition, sustained attention, logical reasoning, working memory, decision-making, and mental quickness abilities. The ADHD of field 302 and the Depression of field 306 may focus on cognitive difficulties associated with different neurobiological phenomena. For example, one mental health or neurocognitive condition, or clinical program, may be associated with a particular network of brain regions, such as dorsal attention network, and one clinical program may be associated with a brain network subserving with the central executive functions. In the present illustration field 301 includes a message for a particular user 1 which facilitates efficient use of the program. For example, the message in field 301 recommends 20 minute exercise sessions four times a week. The user 1 is instructed to use a headphone 50 or speaker 81 in the transducer module 78 to receive stimuli and to tap the touch input 44 to provide responses. The screen 300 may be customized to represent any selection of exercises. In one form, separate apps may be provided, each with a different set of exercises. In another form, a user may interface with a website to select different exercises and have the entity operating the website produce a customized app for download and installation on a smartphone 12. A start button 307 may invoke a “quick start” routine in which a stored sequence is used to initialize operation.

In part “b” of FIG. 8 the screen 300 is replaced by screen 331. This screen 331 provides for performance of an exercise. The exercise field 330 in the present illustration identifies a sequence of letters or numbers which warrant a particular response according to the instructions for the exercise. Field 332 includes a set of instructions for performing the current exercise. A user 1 will hear stimuli in succession. The user 1 is to provide a response per the instructions. Field 332 provides a more detailed set of instructions. For an exercise called Letter Memory Test the instructions in the field 332 reads,

Letter Memory Test is an exercise that trains short-term verbal memory abilities. Short-term verbal memory involves the maintenance of both verbal information over a short period of time. The instructions will describe the target and non-target stimuli in a string of letters that the user will hear. The user will then hear a subsequent set of stimuli. The user will be instructed to make a same or different judgement based on the order of the letters in the string of letters. The user is instructed to perform an action based on the judgement. The action may be performed at touch input 44 of FIG. 1.

Field 334 illustrates the lobes in the brain that are stimulated during the exercise. The present exercise stimulates the frontal lobe and the temporal lobe. This Letter Memory Test exercise is associated with button 304 on the screen 300 in part “a.”

FIG. 9 is a flowchart illustrating performance of the program 20. At block 500 the program developer writes one or more programs and at block 502 provides them to a source such as a server. Each program is associated with a particular cognitive skill. Each program comprises one or more routines. Within each routine, a set of instructions is provided to instruct the user 1 how to respond to stimuli. The steps will comprise a number of repetitions. The program may comprise a combination of routines that have been found to have efficacy in improving cognitive skills in accordance with the present subject matter. At block 510, the user 1 selects a program 20 to perform. The program 20 may have been downloaded prior to the user 1 beginning the program. One convenient way to download a program is to select an app at the iTunes App store or Google Play store and then go to a home screen of the app. At block 512 the user 1 reviews the graphical user interface 76, reading screens that are of interest and selects a routine. The graphical user interface 76 may provide information to assist the user 1 in selecting which routine to perform. One program may contain many routines. One routine may comprise many exercises. Each routine may be directed toward reducing or reversing a corresponding cognitive impairment. At block 516, the processor 40 makes the selection of a desired exercise to perform first. The user 1 may perform more than one exercise during a neural cognitive training session. At block 516 the user 1's selection at block 512 invokes a particular routine in the program memory 34. The program memory 34 contains instructions for indexing each routine through its component exercises, and indexing each exercise through its component steps. At block 520 the program memory 34 provides outputs, which command the components of the currently selected routine.

At block 522 a sequence register responds to the output at block 520 in order to command the components of the currently selected routine. At block 524 stimuli are commanded. The steps are coupled at block 526 to activate transducers in the transducer module 78 (FIG. 6) to produce signals. These produced signals are the stimuli presented to the user 1. At block 528 this system is enabled to receive responses from the user 1. The received signals comprise the user 1's responses.

At block 530 the sequence of events for a current routine is received from the program memory 30 and is stored in a register. The completion of each performed step is registered at block 532. The degree of completion of the steps registered at block 532 is compared to the input from block 530. Block 532 provides an output to block 534 indicative of whether a current routine is completed. At block 534 the indication of whether the current routine is complete is used to select a next step. If the current routine is not complete, operation returns to block 528 where a next step of the current routine is selected. If the routine is completed operation proceeds to block 540, which provides a command to block 520 for initiation of a next routine. The method comprises registering responses from the user and storing results indicative of comparison of user responses to preselected criteria.

At block 540 a timer measures the time between transmission of a stimulus and receipt of a response. If after a preselected time no response is received, the timer circuit may trigger an error signal. This error signal can be used to send a message to the user 1 through the touch screen 83. The message could comprise, “continue?,” “terminate exercise?,” or “restart?.” As stated above, at block 524 the currently completed step is compared to a list of all steps in a routine and an output indicative of whether or not the routine has been completed is viewed at block 534.

Once operation has proceeded to block 532, progress through the sequence of routines is monitored. The progression from block 532 to block 534 provides an indication of whether all routines in an exercise have been completed. If not, operation returns to block 520 where a next routine is selected. If the exercise is completed, operation proceeds to block 540. The results of the exercise are evaluated at block 540. A report such as the report illustrated in panel f of FIG. 7 is produced at block 542. The user 1 is thereby informed of the results of the exercise.

Additionally, at blocks 544 and 546 operations may be performed at a remote location. At block 544 results for a particular user 1 are stored. Processed results can be correlated with exercises performed at block 546. At block 548 a long term medical record may be generated when the user 1 performs a test periodically over an extended time. Further processing is performed at block 550. This processing may include construction of statistical libraries. The statistical data may be used to mine data indicative of many diagnostic features in order to generate knowledge regarding neurocognitive impairment and to customize programs for individual users or groups of users. A library of test results from a population of individual users may be provided for comparison to a selected user's results whereby an evaluation of the selected user's cognitive condition is determined.

FIG. 10 is a chart of cognitive exercises and a description of parameters of the exercises and the portions of the brain engaged. FIG. 10 comprises rows 1 through 10 and columns A through E. The names of cognitive exercises are listed in each row of column A of FIG. 10 and in the first column of Table I below. The cognitive exercises are based on the cognitive exercises having the listed Standardized Neurological Task Names and psychometric parameters, which have been adapted for combination with physical exercise. It has been found that one skilled in the art may customize such parameters, including stimuli type, number of stimuli including targets and non-target configuration, randomization of presentation as the number of repetitions in each exercise and stimulus-response allowable time periods for evaluating responses. The right hand column in Table I lists the references defining each exercise.

TABLE I

| | |
|---|---|
| 1. Simple 4-choice | Wilkinson, R. T., & Houghton, D. (1975). |
| reaction time task | Portable four-choice reaction time test with |
| | magnetic tape memory. Behavior Research |
| | Methods & Instrumentation, 7(5), 441-446. |
| 2. Math Processing | Perez, W. A., Masline, P. J., Ramsey, E. G. and |
| Task | Urban, K. E. (1987). Unified Tri-services |
| | cognitive performance assessment battery: |
| | Review and methodology, DTIC Document |
| | ADA181697 |
| 3. AX-Continuous | Cohen, J. D., Barch, D. M., Carter, C. S., & |
| Performance Test | Servan-Schreiber, D. (1999). Schizophrenic |
| (AX-CPT) | deficits in the processing of context: |
| | Converging evidence from three theoretically |
| | motivated cognitive tasks. Journal of |
| | Abnormal Psychology, 108, 120-133 |
| 4. Sustained Attention | Robertson, I. H., Manly, T., Andrade, J., |
| to Response Task | Baddeley, B. T., Yiend, J. (1997). ‘Oops!’: |
| (SART) | performance correlates of everyday attentional |
| | failures in traumatic brain injured and normal |
| | subjects. Neuropsychologia, 35(6), 747-758. |
| 5. Rapid Visual | Neale C, Johnston P, Hughes M, Scholey A |
| Information Processing | (2015) Functional Activation during the Rapid |
| (RVIP) Tasks | Visual Information Processing Task in a |
| | Middle Aged Cohort: An fMRI Study. PLoS |
| | ONE 10(10): e0138994. |
| | https://doi.org/10.1371/journal.pone.0138994 |
| 6. Corsi Task | Kessels, R. P. C., van Zandvoort, M. J. E., |
| | Postma, A., Kappelle, L. J., & de Haan, E. H. |
| | F. (2000). The Corsi Block-Tapping Task: |
| | Standardization and Normative Data. Applied |
| | Neuropsychology, 7(4), 252-258. |
| 7. N-Back | OKirchner, W. K. (1958). Age differences in |
| | short-term retention of rapidly changing |
| | information. J. Exp. Psychol. 55, 352-358. doi: |
| | 10.1037/h0043688 |
| 8. Letter Memory Test | Inman, Tina Hanlon, Vickery, Chad D., Berry, |
| | David T. R., Lamb, David G., Edwards, |
| | Christopher L., & Smith, Gregory T. (1998). |
| | Development and Initial Validation of a New |
| | Procedure for Evaluating Adequacy of Effort |
| | Given During Neuropsychological Testing: |
| | The Letter Memory Test. Psychological |
| | Assessment, 10(2), 128-39. |
| 9. Reasoning test | Baddeley, A. D. (1968) A 3 min reasoning test |
| based on grammatical | based on grammatical |
| transformation | transformation. Psychon Sci 10, 341-342. |
| | https://doi.org/10.3758/BF03331551 |
| 10. Word/Pseudoword | Xiao Z, Zhang J X, Wang X, Wu R, Hu X, |
| Discrimination Task | Weng X, Tan LH. (2005) Differential activity |
| | in left inferior frontal gyrus for pseudowords |
| | and real words: an event-related fMRI study on |
| | auditory lexical decision. Human Brain |
| | Mapping. 25(2): 212-221. |

In FIG. 10, Columns B through G describe characteristics of each exercise and how they are employed in accordance with the present subject matter. Column B identifies the name of the Genius Gyms Cognitive Task associated with the Standardized Neuropsychological Task. Column C identifies the cognitive domain, namely the predominate aspect of cognition being targeted. Column D describes the particular cognitive skill being trained. Column E describes the type of aural stimuli used in the cognitive task. Column F lists the primary, large-scale neural network trained through the task. Column G is an abbreviated list of the specific brain networks which are engaged by the particular cognitive exercise.

Row 1 describes Simple 4-choice reaction time task. This Genius Gyms exercise, named Parietal Processes, affects the cognitive domain of processing speed. It trains the user 1's information processing speed abilities. Information processing speed is a low level cognitive construct that lies central to most cognitive processes and is how quickly one can process and respond to incoming information. The cognitive task requires the user 1 to make a forced choice response. At the beginning of an exercise the smartphone 12 provides a set of instructions to the user 1. An abbreviated set of instructions may also appear in screen 330 in FIG. 8. The instructions may require the user 1 to provide a response for a target stimulus or a alternate response for a non-target stimulus.

The smartphone 12 instructs the user 1. The instructions tell the user to perform one of a plurality of possible physical actions in response to the stimulus of hearing a respective letter. Each physical action may be performed at an input to the smartphone 12, such as the touch input 44 of FIG. 1. Each stimulus letter has a significant phoneme in common with the other stimuli. Time is measured between issuance of a stimulus and action of the user. There are successive levels of difficulty which are determined by the allotted time. Successive levels of difficulty could be defined by decreases in fractions of a second. The brain regions trained are in the prefrontal cortex, in the left ventral prefrontal cortex, in the temporal lobe, in the superior temporal gyrus, and in the parietal cortex, and occipital cortex, in the occiptotemporal junction.

Row 2 describes Math Processing Task. This Genius Gyms exercise, named Cognitive Calculations, affects the cognitive domain of speed. It trains the user 1's mathematical processing speed abilities. The cognitive task requires the user 1 to perform information processing. Information processing speed is a low level cognitive construct that lies central to most cognitive processes and is how quickly one can process and respond to incoming information. Instructions provided to the user 1 requires the user 1 to perform a calculation and compare the calculation to a threshold. The instructions may require the user 1 to provide a response for a target stimulus or a alternate response for a non-target stimulus. There is a set number of stimuli. A nominal successful response time is selected in milliseconds. The brain regions targeted are the bilateral parietal cortex, dorsolateral and inferior frontal gyri, and the anterior cingulate.

Row 3 exercise is called AX-Continuous Performance Test (AX-CPT). The Genius Gyms task, named Prefrontal Pairs, trains the cognitive domain of attention. The cognitive task requires selective attention/cognitive control context processing and response inhibition. This trains the user 1's selective attention abilities. Response inhibition allows suppression of irrelevant information in working memory to allow access to relevant information.

Selective attention refers to the ability to attend to some stimuli while disregarding others that are irrelevant to the task at hand. The instructions may require the user 1 to provide a response for a target stimulus or an alternate response for a non-target stimulus. Difficulty can be increased with more stimuli in a set or harder responses to remember. The brain regions targeted are dorsolateral prefrontal cortex (DLPFC) and anterior cingulate, posterior visual areas, such as the middle occipital gyrus.

Row 4 exercise is called Sustained Attention to Response Task (SART). The Genius Gyms task, named Focus Finder, trains the cognitive domain of attention. The cognitive task requires maintaining sustained attention for the purpose of training sustained attention abilities. Sustained attention refers to the ability to maintain concentration on a task over an extended period of time and maintain a consistent behavioral response during continuous and repetitive activity. The instructions may require the user 1 to provide a response for a target stimulus or an alternate response for a non-target stimulus. The brain regions affected are frontal cortex, parietal cortex: mainly right sided, dorsomedial, mid- and ventrolateral prefrontal cortex, anterior insula, parietal areas (intraparietal sulcus, temporo-parietal junction), and subcortical structures (cerebellar vermis, thalamus, putamen, midbrain).

Row 5 exercise is called Rapid Visual Information Processing (RVIP) Task. The Genius Gyms task is named Rapid Response, and trains the relevant cognitive domain of attention. The cognitive task requires maintaining sustained attention for the purpose of training sustained attention abilities. Sustained attention refers to the ability to maintain concentration on a task over an extended period of time and maintain a consistent behavioral response during continuous and repetitive activity. The instructions may require the user 1 to respond whether or not the stimuli comprise a target sequence. The affected regions of the brain are frontal cortex, parietal cortex, and cerebellum.

Row 6 exercise is called Corsi Task. The Genius Gyms task is named Galileo's Clocks trains the relevant cognitive domain of memory. The cognitive task is using visual-spatial stimuli for the purpose of training short-term memory. Short-term visuospatial memory involves the maintenance of both visual and spatial information over a short period of time. The instructions may require the user 1 to provide one of a plurality of available responses based on sequences of pseudorandom numbers. The instructions may require the user 1 to respond after as to whether the order of stimuli in a group are repeated correctly or incorrectly. The brain regions affected dorsolateral prefrontal (BA Aug. 9, 1946) and ventrolateral prefrontal (BA 47), bilateral parietal lobes (BA 7/40), bilateral hippocampus, cingulate gyrus, thalamus, caudate and cerebellum.

Row 7 exercise is N-Back. The Genius Gyms task is named Recall Repeats and trains the relevant cognitive domain of memory. This cognitive task trains working memory abilities. Working memory is a multidimensional cognitive construct that has been hypothesized as the fundamental source of age-related deficits in a variety of cognitive tasks, including long-term memory, language, problem solving, and decision making. The instructions may require the user 1 to provide a different response to whether the stimulus is a target or non-target in a string of letters. The brain regions affected are the frontal cortex, in the left dorsolateral prefrontal cortex and parietal lobe.

Row 8 is Letter Memory Test. The Genius Gyms task is named Memory Match and trains the relevant cognitive domain of short-term verbal memory. Short-term verbal memory involves the maintenance of both verbal information over a short period of time. The cognitive task trains the cognitive skills associated with short-term verbal memory. The instructions may require the user 1 to make a same or different judgment about a corresponding string of letters. The brain region affected is the dorsolateral prefrontal cortex.

Row 9 exercise is Reasoning test based on grammatical transformation test. The Genius Gyms task is named Descartes' Decisions and the relevant trained cognitive domain is reasoning. The task trains logical reasoning abilities. Reasoning is a cognitive construct within executive control and is a multi-component construct that consists of a range of different processes that are involved in the planning, organization, coordination, implementation, and evaluation of many of our nonroutine activities. The instructions may require the user 1 to respond after as to whether the order of stimuli in a group are repeated correctly or incorrectly.

Row 10 exercise is Word/Pseudoword Discrimination Task. The Genius Gyms task is named Wernicke's Words and trains the relevant cognitive domain of reasoning. The task trains lexical decision making and the cognitive task is training lexical decision making abilities. Decision-making is a cognitive domain that makes significant demands on processing resources, but in everyday life those demands may be reduced by life-relevant knowledge or expertise in the problem-solving domain. The instructions may require the user 1 to provide a response for a target stimulus or an alternate response for a non-target stimulus. The brain region affected is the left temporal lobe, within Wernicke's area, which is a language comprehension area.

In accordance with the present subject matter, these exercises are combined in specific combinations. As described above, each exercise interacts with a selected part or parts of the brain. By combining selected exercises in a particular sequence or sequences, selected neural circuits are engaged. This method comprises a battery of exercises which invoke neural circuits for a clinical disorder in which the method defines a set of cognitive skills associated with a clinical disorder and commands performance of a preselected battery of cognitive exercises associated with each cognitive skill.

By providing the particular routines, each selected cognitive impairment may be improved. Although prior combinations of physical and cognitive exercise have been used to delay or prevent onset of dementia, there have not been provided specific combinations of exercises into routines in order to engage brain circuits to address a selected cognitive impairment. This method includes associating specific cognitive impairments with specific routines. This method has aided users in not just postponing cognitive impairments, but improving selective cognitive abilities. Users with no current cognitive impairment may use the current method and system for improved and lasting cognitive help. This method utilizes an intricate understanding of the functions of the brain connected to each cognitive ability. The combination with physical exercise reacts synergistically with the cognitive program. In the present method, aerobic exercise is preferred, but is not essential. Audio stimuli are also preferred, but not essential. The advantage of audio stimuli is that they allow concentration to be focused on cognitive tasks that would otherwise be devoted to concentrating on physical surroundings.

Exercise programs may also be provided that comprise a “randomized” group of exercises, called “Quick Start.” In one form ordering the designated cognitive skill exercises comprises selecting a time period for performing exercises and providing one of a preselected set of routines and establishing a preselected time duration for performing each exercise. For example, for a five minute exercise a processor may be directed to select three exercises randomly out of a list of available exercises. For a twenty minute walk, seven exercises may be performed in twenty minutes.

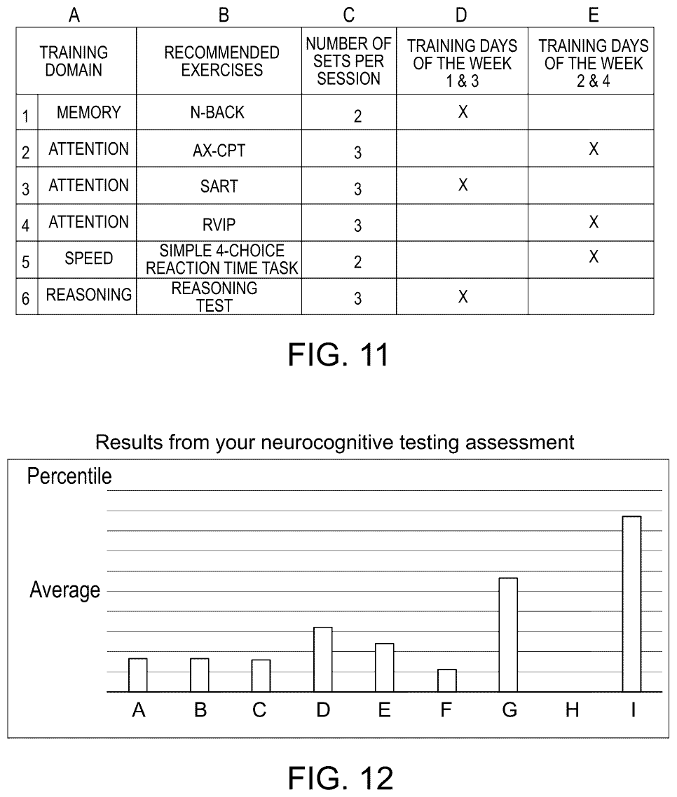

FIG. 11 is a nominal program individualized to a specific user 1. The chart in FIG. 11 comprises columns A through E and rows 1 through 6. In column A the training domain is listed. A training domain is a particular cognitive skill. Cognitive skills include abilities such as memory, attention, speed, and reasoning. This training program associates particular exercises with corresponding neurocognitive skills. Each exercise is an established set of actions. N-Back is an exercise in which a user 1 must perceive whether a particular letter stimulus matches a previous stimulus remembered by the user 1. Columns C, D, and E define a training schedule over a four week period. The training schedule may be a fixed training schedule suggested to all users. Alternatively, the training schedule may be personalized to a particular user based on results achieved with previous exercise regimens. Column C lays out the number of sets per session to be performed. Column D identifies the training days of the first and third weeks of the 4 week regimen. Column E identifies the training days of the second and fourth weeks of the 4 week regimen. This chart demonstrates a specific embodiment of a routine utilizing the teachings in FIG. 10.

Training for the memory domain includes performing the N-Back exercise using two sets per session of the exercise on training days 1 and 3 of training weeks 1 and 3. The other cognitive skill exercises each have recommended number of sets per session and specified training days. In order to address the neurocognitive skill of attention, there are 3 forms of recommended exercise. These exercises are labeled AX-Continuous Performance Test (AX-CPT), Sustained Attention to Response Task (SART), and Rapid Visual Information Processing (RVIP) Task. Addressing speed uses an exercise called Simple 4-choice reaction time task. Here again there is a number of sets per session and training days are identified. Dealing with reasoning the exercise used is Reasoning test based on grammatical transformation. Again here the number of sets per session are listed and the days of the week for training are identified. Users can be evaluated for determination of a recommended set of training domains and particular exercises.

All of the routines and exercises in the present method are designed to activate a specific network of cognitive processes. Directed programs include routines which are directed toward creating a set of cognitive exercises which when taken together address a respective cognitive impairment or address a set of skills for use by a preselected population.

In accordance with the present subject matter, the following cognitive impairments are addressed by corresponding set of exercise as follows:
Attention-deficit/hyperactivity disorder (ADHD)⇒AX-Continuous Performance Test (AX-CPT), Sustained Attention to Response Task (SART), Rapid Visual Information Processing (RVIP) Task, Simple 4-choice reaction time task, Math Processing Task, Word/Pseudoword Discrimination Task
Addiction⇒AX-Continuous Performance Test (AX-CPT), Reasoning Test Based on Grammatical Transformation, Word/Pseudoword Discrimination Task, N-back, Sustained Attention to Response Task (SART), Simple 4-choice reaction time task
Anxiety⇒AX-Continuous Performance Test (AX-CPT), Corsi Task, Word/Pseudoword Discrimination Task, N-back, Math Processing Task, and Letter Memory Task
Autism⇒AX-Continuous Performance Test (AX-CPT), Rapid Visual Information Processing (RVIP) Task, Reasoning Test Based on Grammatical Transformation, Word/Pseudoword Discrimination Task, Simple 4-choice reaction time task, Math Processing Task
Brain Fog⇒Sustained Attention to Response Task (SART), Math Processing Task, Simple 4-choice reaction time task, N-back, Reasoning Test Based on Grammatical Transformation, Rapid Visual Information Processing (RVIP) Task
Cognitive Aging⇒N-Back, Letter Memory Test, Math Processing Task, Sustained Attention to Response Task (SART), Word/Pseudoword Discrimination Task, Rapid Visual Information Processing (RVIP) Task
Depression⇒AX-Continuous Performance Test (AX-CPT), Sustained Attention to Response Task (SART), Rapid Visual Information Processing (RVIP) Task, Reasoning Test Based on Grammatical Transformation, Word/Pseudoword Discrimination Task, Math Processing Task
Multiple Sclerosis⇒Simple 4-choice reaction time task, Math Processing Task, Letter Memory Test, Corsi Task, Wernicke's Words, Sustained Attention to Response Task (SART)
Obsessive compulsive disorder (OCD)⇒Reasoning Test Based on Grammatical Transformation, Corsi Task, Simple 4-choice reaction time task, Math Processing Task, Word/Pseudoword Discrimination Task, AX-Continuous Performance Test (AX-CPT)
Pain⇒Sustained Attention to Response Task (SART), Rapid Visual Information Processing (RVIP) Task, AX-Continuous Performance Test (AX-CPT), N-back task, Word/Pseudoword Discrimination Task, Math Processing Task
Parkinson's Disease⇒AX-Continuous Performance Test (AX-CPT), Rapid Visual Information Processing (RVIP) Task, N-Back, Simple 4-choice reaction time task, word/pseudoword discrimination task
Post-Traumatic Stress Disorder (PTSD)⇒Reasoning Test Based on Grammatical Transformation, AX-Continuous Performance Test (AX-CPT), Letter Memory Test, Sustained Attention to Response Task (SART), Corsi Task, Word/Pseudoword Discrimination Task
Schizophrenia⇒AX-Continuous Performance Test (AX-CPT), Simple 4-choice reaction time task, N-back, Letter Memory Test, Reasoning Test Based on Grammatical Transformation, Corsi Task
Stress⇒N-Back, Letter Memory Test, Corsi Task, Reasoning Test Based on Grammatical Transformation, Word/Pseudoword Discrimination Task, AX-Continuous Performance Test (AX-CPT)

A plurality of “Optimizer Programs” have been developed. Each program combines specific exercises to support and strengthen skills for specific groups of users.
Team Athletes⇒Reasoning Test Based on Grammatical Transformation, Corsi Task, Simple 4-choice reaction time task, Math Processing Task, Sustained Attention to Response Task (SART), AX-Continuous Performance Test (AX-CPT)
Executives⇒Reasoning Test Based on Grammatical Transformation, Math Processing Task, Simple 4-choice reaction time task, Corsi Task, Rapid Visual Information Processing (RVIP) Task, Word/Pseudoword Discrimination Task
Golfer⇒AX-Continuous Performance Test (AX-CPT), Sustained Attention to Response Task (SART), Rapid Visual Information Processing (RVIP) Task, Corsi Task, Simple 4-choice reaction time task, Letter Memory Test
Gamer⇒Corsi Task, Sustained Attention to Response Task (SART), Rapid Visual Information Processing (RVIP) Task, Simple 4-choice reaction time task, AX-Continuous Performance Test (AX-CPT)
Other routines that have been developed and their component exercises are listed below: “Brain Breaks—Small cognitive domain specific training programs with only 3 exercises in the program
“Get Focused” Attention Training (current name: Attention): AX-Continuous Performance Test (AX-CPT), Sustained Attention to Response Task (SART), Rapid Visual Information Processing (RVIP) Task
“Memory Boost” Memory Training: N-Back, Letter Memory Test, Corsi Task
“Speed up” Processing Speed Training: Simple 4-choice reaction time task, Math Processing Task, Sustained Attention to Response Task (SART)
“Quick Decisions” Decision-Making and reasoning training: Reasoning Test Based on Grammatical Transformation, Word/Pseudoword Discrimination Task, Math Processing Task

FIG. 12 illustrates a comprehensive neurocognitive testing assessment that has been generated by a third party. The user takes this third party cognitive testing assessment. The practitioner of the present method uses the data to personalize a program for that user. This figure is a chart showing a neurocognitive assessment of an individual. It demonstrates how an induvial ranks in comparison to a tested population. The tested population may be represented by a statistical body as produced at block 550 of FIG. 9. This individual's assessment shows that memory is at various points below average. The bars in columns A, B, and C show the percentile rank for different memory skills, such as working memory. The bars in columns D and E are higher than below average and roughly in the 30^{th} percentile. These are measures of attention such as cognitive control. The bars in columns F and G represent speed skills. Column F which is processing speed is at below average percentile. Column G which also relates to speed is at average percentile. The bars in columns H and I represent measures of reasoning. Column H is empty. Column I is approaching above average. These measurements are used to provide benchmarks for comparison to the results of other tests. Statistical libraries of tests will be used in the future to provide cognitive measures not yet known or identified.

FIG. 13 is a chart of clinical disorders that are associated with specific neural circuits and cognitive skills associated with selected disorders. A listing of clinical disorders is found on FIG. 13. Exercises from the above teachings may be applied to address the cognitive impairments associated with the disorder. Representative selected clinical disorders are listed with a further description of symptoms of the disorder. This information can be used in accordance with the present subject matter to address these clinical disorders.

Although the foregoing description has specified certain steps, exercises, routines and programs that may be used in the method of the present invention, those skilled in the art will appreciate that many modifications and substitutions may be made. While a number of programs and routines have been articulated, they are presented as being exemplary and not limiting. While the invention has been described in terms of several embodiments, those of ordinary skill in the art will recognize that the invention is not limited to the embodiments described, but can be practiced with modification and alteration within the spirit and scope of the appended claims.

1. A method for remediating Depression comprising engaging specific neural circuits through the simultaneous performance of physical exercise and cognitive training exercises, wherein the cognitive training exercises are delivered to a user via a device that presents mental stimuli while the user is engaged in physical exercise, and wherein the user provides responses to the mental stimuli during the physical exercise that are received and evaluated with respect to criteria associated with the respective cognitive exercise, such that the simultaneous performance of the physical and cognitive components induces synergistic activation of neural circuits and elicits activation of the targeted brain regions through engagement in the cognitive training exercises; wherein the steps comprise:
performing an AX-Continuous Performance Test (AX-CPT) exercise for training inhibition and disengagement to engage the dorsolateral prefrontal cortex (DLPFC) and anterior cingulate cortex (ACC) cognitive control nodes;
performing a Sustained Attention to Response Task (SART) exercise for maintaining task focus to counter rumination to engage prefrontal, parietal, and cerebellar attentional control networks;
performing a Rapid Visual Information Processing (RVIP) exercise for building sustained attention and vigilance and eliciting disengagement of ruminative thoughts to engage bilateral fronto-parietal-cerebellar networks;
performing a Reasoning (grammatical) exercise for exercising top-down control, specifically to override hyperactive limbic circuits to subgenual ACC to prefrontal cortex and reappraisal processes to engage frontal and parietal regions, the cingulate cortex, caudate and insula;
performing a Word/Pseudoword Lexical Decision exercise for training rapid, non-emotional lexical selection to reduce biased processing to engage frontal-temporal decision and language regions; and
performing a Math Processing exercise for enhancing processing speed and controlled cognitive operations to engage DLPFC, inferior frontal gyrus (IFG), ACC, and parietal regions.

2. A method for remediating Cognitive Aging comprising engaging specific neural circuits through the simultaneous performance of physical exercise and cognitive training exercises, wherein the cognitive training exercises are delivered to a user via a device that presents mental stimuli while the user is engaged in physical exercise, and wherein the user provides responses to the mental stimuli during the physical exercise that are received and evaluated with respect to criteria associated with the respective cognitive exercise, such that the simultaneous performance of the physical and cognitive components induces synergistic activation of neural circuits and elicits activation of the targeted brain regions through engagement in the cognitive training exercises; wherein the steps comprise:
performing an N-back exercise for challenging and improving working memory to engage the left dorsolateral prefrontal cortex (DLPFC), cingulate cortex, and parietal regions;
performing a Letter Memory exercise for improving short-term verbal memory to engage the medial temporal lobe, including the hippocampus, and prefrontal and parietal regions;
performing a Math Processing exercise for improving processing speed and numerical reasoning fluency to engage frontal-parietal working memory circuits, and specifically engaging the DLPFC, IFG and ACC;
performing a Sustained Attention to Response Task (SART) exercise for reinforcing sustained attention and reducing attentional drift to engage the prefrontal cortex, including ACC, and parietal regions,
performing a Word/Pseudoword Lexical Decision exercise for analyzing time and accuracy of lexical and pseudolexical decisions to engage the left occipito-temporal sulcus, left superior temporal gyrus, the left inferior frontal gyrus, left supramarginal gyrus, and the anterior cingulate cortex (ACC), fronto-parietal networks, and domain-general multiple-demand regions for decision processes;
and performing a Rapid Visual Information Processing (RVIP) exercise for improving the speed and accuracy of processing visual information to engage bilateral fronto-parietal-cerebellar neural networks.

3. A method for remediating Attention-Deficit/Hyperactivity Disorder (ADHD) comprising engaging specific neural circuits through the simultaneous performance of physical exercise and cognitive training exercises, wherein the cognitive training exercises are delivered to a user via a device that presents mental stimuli while the user is engaged in physical exercise, and wherein the user provides responses to the mental stimuli during the physical exercise that are received and evaluated with respect to criteria associated with the respective cognitive exercise, such that the simultaneous performance of the physical and cognitive components induces synergistic activation of neural circuits and elicits activation of the targeted brain regions through engagement in the cognitive training exercises; wherein the steps comprise:
performing an AX-Continuous Performance Test (AX-CPT) exercise for improving response inhibition and selective attention to combat arousal/control issues by engaging the dorsolateral prefrontal cortex (DLPFC) and anterior cingulate cortex (ACC), posterior visual (middle occipital gyrus);
performing a Sustained Attention to Response Task (SART) exercise for improving sustained attention to engage right dorsomedial and ventrolateral prefrontal cortex (dmPFC/vIPFC), anterior insula, inferior parietal sulcus, temporoparietal junction, cerebellar vermis, thalamus, putamen, and midbrain structures;
performing a Rapid Visual Information Processing (RVIP) exercise for enhancing sustained attention to engage frontal, parietal, and cerebellar regions within fronto-parietal-cerebellar networks;
performing a Simple 4-choice reaction time task (S4CRTT) exercise for fast and accurate decision-making by increasing information processing speed by engaging left ventral prefrontal cortex, superior temporal gyrus, parietal cortex, and occipito-temporal junction;
performing a Math Processing exercise for improving processing speed and effort allocation to engage bilateral parietal cortex, DLPFC, inferior frontal gyrus (IFG), and ACC; and
performing a Word/Pseudoword Lexical Decision exercise for improving rapid lexical rule-based decision-making and selection/inhibition demands that exercise cognitive control processes to engage left temporal language areas, including Wernicke's area, and fronto-temporal decision regions.

4. A method for remediating Anxiety Disorder comprising engaging specific neural circuits through the simultaneous performance of physical exercise and cognitive training exercises, wherein the cognitive training exercises are delivered to a user via a device that presents mental stimuli while the user is engaged in physical exercise, and wherein the user provides responses to the mental stimuli during the physical exercise that are received and evaluated with respect to criteria associated with the respective cognitive exercise, such that the simultaneous performance of the physical and cognitive components induces synergistic activation of neural circuits and elicits activation of the targeted brain regions through engagement in the cognitive training exercises; wherein the steps comprise:
performing an AX-Continuous Performance Test (AX-CPT) exercise for improving response inhibition and selective attention to control excessive worrying and ability to elicit top-down control of the limbic activation by engaging the dorsolateral prefrontal cortex (DLPFC) and anterior cingulate cortex (ACC);
performing a Corsi visuospatial memory span exercise for improving short-term visuospatial memory and attention integration to engage the DLPFC, ventrolateral prefrontal cortex (VLPFC), parietal cortex, hippocampus, cingulate cortex, thalamus, caudate, and cerebellum;
performing a Word/Pseudoword Lexical Decision exercise for training rapid, non-emotional lexical selection to counter biased verbal processing to engage left temporal cortex (Wernicke's area) and associated fronto-temporal language decision regions;
performing an N-back working memory exercise for strengthening working memory control, which is central to the regulation of thoughts, to engage DLPFC, cingulate cortex, and parietal regions;
performing a Math Processing exercise for improving processing speed and controlled mental operations to engage bilateral parietal regions, DLPFC, inferior frontal gyrus (IFG), and ACC;
and performing a Letter Memory exercise for improving short-term verbal memory maintenance supporting self-referential regulation to engage the DLPFC and medial temporal lobe.

5. A method for treating a cognitive disorder from the group consisting of Attention Deficit Hyperactivity Disorder (ADHD), addiction, anxiety, autism, brain fog, cognitive aging, depression, Multiple Sclerosis (MS), Obsessive Compulsive Disorder (OCD), Parkinson's Disease, pain, Post Traumatic Stress Disorder (PTSD), Schizophrenia, and stress, whereby specific neural circuits are engaged in sets of specific timed exercise tasks with each said set of exercises being correlated to specifically remediate with one said cognitive disorder, the exercises each activating specific neural circuits for engaging a respective cognitive function in said cognitive disorder;
a. wherein the cognitive disorder is ADHD and the neural circuits to target comprise subcortical-thalamocortical and cerebellar-frontal loops, arousal systems including locus coeruleus, basal forebrain, intralaminar thalamus, right PFC, and cerebellum, and wherein the set of six cognitive exercises is configured to remediate cognitive control, working memory, inhibition, response/arousal regulation, and planning inefficiency deficits, the set comprising AX-Continuous Performance Test (AX-CPT), Sustained Attention to Response Task (SART), Rapid Visual Information Processing (RVIP) Task, Simple 4-choice reaction time task (S4CRTT), Math Processing Task, and Word/Pseudoword Lexical Decision Task, wherein AX-CPT is configured to train inhibition and selective attention via DLPFC/ACC engagement, SART and RVIP are configured to strengthen sustained attention across fronto-parietal-cerebellar networks, S4CRTT and Math Processing Task are configured to improve processing speed across PFC-parietal-temporal hubs, and Word/Pseudoword is configured to exercise lexical decision-making to enhance selection/inhibition control via engaging the fronto-temporal brain region;
b. wherein the cognitive disorder is addiction and the neural circuits to target nucleus accumbens, dorsal striatum, PFC and ACC, and wherein the set of six cognitive exercises is configured to remediate impaired response inhibition, cognitive-behavioral control, working memory, attention, reasoning, decision-making deficits, the set comprising AX-Continuous Performance Test (AX-CPT), Reasoning Test Based on Grammatical Transformation, N-back, Sustained Attention to Response Task (SART), Simple 4-choice reaction time task (S4CRTT), and Word/Pseudoword Lexical Decision Task, wherein AX-CPT is configured to train response inhibition and selective attention via DLPFC and ACC engagement along with activating fronto-parietal networks, Reasoning Test Based on Grammatical Transformation strengthens reasoning/decision-making demands while recruiting frontal-striatal and cingulate control hubs, N-back is configured to train working memory deficits and recruits the DLPFC and cingulate control network brain regions, SART is configured to strengthen sustained attention across fronto-parietal-cerebellar networks, S4CRTT is configured to improve processing speed across PFC-parietal-temporal hubs, and Word/Pseudoword is configured to exercise lexical decision-making to enhance selection/inhibition control via engaging the fronto-temporal brain regions;
c. wherein the cognitive disorder is anxiety and the neural circuits to target comprise amygdala, ACC, temporal/insular cortices, dorsal/ventral PFC, and dysregulated default-mode, salience, and executive networks, and wherein the set of six cognitive exercises is configured to remediate excessive worry, impaired self-regulation, attention reallocation, memory, relational integration, and emotion/cognition integration deficits, the set comprising AX-CPT, Corsi, Word/Pseudoword Lexical Decision Task, N-back, Math Processing, and Letter Memory, wherein AX-CPT is configured to engage DLPFC/ACC to control worry, Corsi is configured to support short-term memory and attention integration via executive-temporal-cingulate nodes, Word/Pseudoword is configured to train fast non-emotional selection to counter biased verbal processing through engaging frontal-temporal brain regions, N-back is configured to strengthen working memory control which is central to regulation of thoughts via DLPFC, cingulate and parietal cortices, Math Processing is configured to improve and build faster, controlled processing speed via frontal and parietal networks, and Letter Memory is configured to improve short-term verbal memory maintenance supporting self-referential system regulation through engagement of the fronto-temporal brain regions, including DLPFC and medial temporal lobe;
d. wherein the cognitive disorder is autism and the neural circuits to target comprise fronto-temporo-parietal networks, right insula/left cingulate, right middle frontal gyrus, and peri-genual and dorsal ACC, and wherein the set of six cognitive exercises is configured to remediate social communication, restricted/repetitive behaviors, and deficits in executive function, motor, visual, auditory-language, and complex cognition, the set comprising AX-CPT, RVIP, Reasoning (grammatical), Word/Pseudoword Lexical Decision Task, Simple 4-choice RT, and Math Processing, wherein AX-CPT is configured to train inhibition/selection via fronto-partietal networks, including ACC/DLPFC, RVIP is configured to support sustained attention within fronto-parietal-cerebellar systems, Reasoning is configured to train complex cognition, such as logical reasoning, via insula and cingulate recruitment, Word/Pseudoword is configured to train language-based decision processes engaging fronto-temporal brain regions, Simple 4-choice RT is configured to improve processing speed across cortical hubs (including within the frontal, parietal and temporal cortices), and Math Processing is configured to strengthen computational quickness via DLPFC, ACC and IFG;
e. wherein the cognitive disorder is brain fog and the neural circuits to target comprise PFC and hippocampus, and wherein the set of six cognitive exercises is configured to remediate impaired processing speed, working memory, visual/verbal learning and memory, and attention, the set comprising SART, Math Processing, Simple 4-choice RT, N-back, Reasoning (grammatical), and RVIP, wherein SART and RVIP are configured to strengthen sustained attention through fronto-parietal-cerebellar activation, Math Processing and Simple 4-choice RT are configured to improve information processing speed using fronto-parietal brain regions (including the PFC), N-back is configured to target working memory via DLPFC and cingulate engagement, and Reasoning is configured to support higher-order cognitive control processes across frontal-parietal networks (including frontal gyri, cingulate and inferior parietal lobe);
f. wherein the cognitive disorder is cognitive aging and the neural circuits to target comprise prefrontal cortex and medial temporal lobe (including hippocampus), and wherein the set of six cognitive exercises is configured to remediate declines in attention, task-switching, working memory, short-term memory, language, decision-making, processing speed, and inhibitory control, the set comprising N-back, Letter Memory, Math Processing, SART, Word/Pseudoword Lexical Decision Task, and RVIP, wherein N-back is configured to train working memory via DLPFC, along with cingulate and parietal brain region engagement, Letter Memory is configured to target short-term verbal memory engaging medial temporal nodes (such as the hippocampus), Math Processing is configured to improve processing speed via frontal control regions and also parietal lobe engagement, SART and RVIP are configured to reinforce sustained attention through activating fronto-parietal neural networks, and Word/Pseudoword is configured to support language-based decision processes through fronto-temporal lobe engagement;
g. wherein the cognitive disorder is depression and the neural circuits to target comprise hyperactive limbic circuits to subgenual/ACC to PFC, reduced top-down control via DLPFC/VLPFC and right superior parietal, and normalizing amygdala, ACC, thalamus, nucleus accumbens, caudate, and hippocampus activity, and wherein the set of six cognitive exercises is configured to remediate negative attentional bias, impaired disengagement/inhibition, rumination, and biased memory/processing, the set comprising AX-CPT, SART, RVIP, Reasoning (grammatical), Word/Pseudoword Lexical Decision Task, and Math Processing, wherein AX-CPT is configured to train inhibition/disengagement via DLPFC/ACC cognitive control nodes, SART and RVIP are configured to maintain task focus to counter rumination via fronto-parietal-cerebellar engagement, Reasoning is configured to exercise top-down control via frontal-parietal networks and cingulate activation, Word/Pseudoword is configured to train rapid non-emotional selection to reduce biased processing utilizing fronto-temporal engagement, and Math Processing is configured to enhance processing speed via DLPFC, IFG, ACC and parietal lobe engagement;
h. wherein the cognitive disorder is MS and the neural circuits to target comprise network dynamics, including default-mode, fronto-parietal, visual networks, and lateralized ACC relationships, and wherein the set of six cognitive exercises is configured to remediate executive function (fluency), processing speed, sustained attention, and memory deficits, the set comprising Simple 4-choice RT, Math Processing, Letter Memory, Corsi, Word/Pseudoword Lexical Decision Task, and SART, wherein Simple 4-choice RT and Math Processing are configured to improve processing speed through frontal-parietal activation tied to executive functioning, Letter Memory and Corsi are configured to train verbal and visuospatial short-term memory (respectively) via frontal-temporal and fronto-parietal-temporal networks (respectively), Word/Pseudoword is configured to support decision/fluency and executive functioning through fronto-temporal engagement, and SART is configured to build sustained attention within fronto-parietal control networks disturbed in MS;
i. wherein the cognitive disorder is OCD and the neural circuits to target comprise limbic frontal-basal ganglia loops, ACC, orbitofrontal cortex, and wherein the set of six cognitive exercises is configured to remediate memory, especially episodic, attention, especially selective, flexibility, inhibition, planning, decision-making, reasoning, and fluency deficits, the set comprising Reasoning (grammatical), Corsi, Simple 4-choice RT, Math Processing, Word/Pseudoword Lexical Decision Task, and AX-CPT, wherein Reasoning is configured to train decision-making via frontal-parietal, cingulate and basal ganglia, specifically caudate, circuits, Corsi is configured to target visuospatial short term memory and organization via fronto-temporal (hippocampal) basal ganglia nodes, Simple 4-choice RT and Math Processing are configured to improve information processing speed through fronto-parietal engagement, Word/Pseudoword is configured to train verbal encoding and selection processes via fronto-temporal activation, and AX-CPT is configured to enhance response inhibition and selective attention via DLPFC and ACC-centered cognitive control loops;
j. wherein the cognitive disorder is pain and the neural circuits to target comprise the pain matrix including lateral and medial components in the frontal lobe, ACC, insula, amygdala, hypothalamus, and peri-aquaeductal grey matter (PAG), and wherein the set of six cognitive exercises is configured to remediate attentional and executive control deficits (decision-making, inhibition, flexibility, adaptability), the set comprising SART, RVIP, AX-CPT, N-back, Word/Pseudoword Lexical Decision Task, and Math Processing, wherein SART and RVIP are configured to strengthen sustained attention and monitoring by engaging the insula and thalamo-frontal nodes, AX-CPT is configured to train response inhibition and selective attention via ACC and DLPFC control network nodes processing, N-back is configured to train working memory through reinforcing executive maintenance during nociceptive distraction, Word/Pseudoword is configured to support decision-making and goal-directed cognition through engagement of fronto-temporal regions, and Math Processing is configured to improve rapid controlled processing through fronto-parietal regional engagement;
k. wherein the cognitive disorder is Parkinson's Disease and the neural circuits to target comprise basal ganglia dopaminergic pathways, nigro-striatum-thalamus-cortical circuit (linking the striatum to the PFC), and cholinergic projections to PFC, and wherein the set of six cognitive exercises is configured to remediate attention, executive function, memory, and visuospatial processing deficits, the set comprising AX-CPT, RVIP, Reasoning (grammatical), N-back, Simple 4-choice RT, and Word/Pseudoword Lexical Decision Task, wherein AX-CPT and Reasoning are configured to train executive control via fronto-striatal and cingulate nodes, RVIP is configured to support sustained attention through activating fronto-parietal-cerebellar control networks, N-back is configured to train working memory through DLFPC involvement, Simple 4-choice RT is configured to improve processing speed affecting daily decision making by engaging fronto-parietal brain regions, and Word/Pseudoword is configured to enhance rapid language-based decision-making and executive function by engaging fronto-temporal networks;
l. wherein the cognitive disorder is PTSD and the neural circuits to target comprise hyperactive amygdala/limbic circuits, reduced PFC, hippocampal and basal ganglia networks involved in conditioning/extinction, and wherein the set of six cognitive exercises is configured to remediate memory, attention, planning, error monitoring, inhibition, and problem-solving deficits, the set comprising Reasoning (grammatical), AX-CPT, Letter Memory, SART, Corsi, and Word/Pseudoword Lexical Decision Task, wherein Reasoning is configured to train problem-solving and error-monitoring via frontal-cingulate networks, AX-CPT is configured to enhance inhibitory control through engaging fronto-parietal brain networks (including DLPFC and ACC), Letter Memory and Corsi are configured to target short-term verbal and visuospatial memory with fronto-temporal engagement and hippocampal-dependent memory maintenance, SART is configured to build sustained attention to counter intrusions through fronto-parietal-cerebellar engagement, and Word/Pseudoword is configured to reinforce rapid non-emotional response selection processes through fronto-temporal engagement;
m. wherein the cognitive disorder is Schizophrenia and the neural circuits to target comprise fronto-parietal hypoconnectivity, abnormal activity in DLPFC, ACC, insula, amygdala, hippocampus, ventral striatum, thalamus, cerebellum, and fronto-parietal connectivity, and wherein the set of six cognitive exercises is configured to remediate processing speed, attention, working memory, learning, declarative memory, executive function, inhibition, cognitive control, and visual/perceptual processing deficits, the set comprising AX-CPT, Simple 4-choice RT, N-back, Letter Memory, Reasoning (grammatical), and Corsi, wherein AX-CPT is configured to train attention/control and response inhibition via fronto-parietal networks, including the DLPFC and ACC brain regions, Simple 4-choice RT is configured to improve processing speed by activating fronto-parietal networks, N-back is configured to strengthen working memory via DLPFC engagement, Letter Memory is configured to support declarative and short-term memory by activating fronto-temporal brain regions, specifically in the medial temporal lobe, Reasoning is configured to exercise executive functioning and logical reasoning via cognitive control networks within fronto-parietal-temporal brain regions, and Corsi is configured to target visuospatial memory and learning via engagement of frontal, cingulate medial temporal and thalamic nodes; and
n. wherein the cognitive disorder is stress and the neural circuits to target comprise insula, striatum, cingulate, SFG/MFG-striatum networks, and right superior temporal gyrus, and wherein the set of six cognitive exercises is configured to remediate immediate attention and cognitive control deficits, particularly inhibitory control, decision-making, and reasoning, the set comprising N-back, Letter Memory, Corsi, Reasoning (grammatical), Word/Pseudoword Lexical Decision Task, and AX-CPT, wherein N-back and Letter Memory are configured to train working and short-term memory (respectively) via fronto-parietal-temporal network, specifically DLPFC and cingulate, activation, Corsi is configured to build visuospatial short-term memory engaging striatal and cingulate nodes along with a core brain region affected by stress (hippocampus), Reasoning is configured to train decision-making with engagement of sub-cortical insula and caudate and frontal-parietal activity in building stress-resilient networks, Word/Pseudoword is configured to train rapid controlled response selection through fronto-temporal engagement, and AX-CPT is configured to target inhibitory control and selective attention by activating regions affected by stress including the prefrontal cortex and parietal lobe.
